(19) United States
(12) Patent Application Publication
(10) Pub. No.: US 2025/0258431 A1
(43) Pub. Date: Aug. 14, 2025

(54) RADIATION-SENSITIVE COMPOSITION, METHOD FOR FORMING RESIST PATTERN, POLYMER, AND COMPOUND

(71) Applicant: JSR CORPORATION, Tokyo (JP)

(72) Inventors: Ken MARUYAMA, Tokyo (JP); Katsuaki NISHIKORI, Tokyo (JP); Takahiro KAWAI, Tokyo (JP)

(73) Assignee: JSR CORPORATION, Tokyo (JP)

(21) Appl. No.: 19/044,756

(22) Filed: Feb. 4, 2025

(30) Foreign Application Priority Data
Feb. 9, 2024 (JP) 2024-018734
Dec. 11, 2024 (JP) 2024-216987

(51) Int. Cl. G03F 7/039; C07C 25/18; C07C 309/12; C07C 323/09; C08F 212/14; C08F 220/18; C08F 220/22; C08F 220/40; G03F 7/004; G03F 7/038

(52) U.S. Cl. CPC G03F 7/0397; C07C 25/18; C07C 309/12; C07C 323/09; C08F 212/22; C08F 220/1806; C08F 220/1812; C08F 220/22; C08F 220/40; G03F 7/0046; G03F 7/0382; G03F 7/0388; C07C 2603/88; C08F 2800/10

(57) ABSTRACT

A radiation-sensitive composition contains a polymer having a partial structure represented by formula (1), and a radiation-sensitive acid generating substance. The radiation-sensitive composition satisfies one or more of requirement 1, requirement 2, and requirement 3. requirement 1: the partial structure represented by the formula (1) has two or more iodine atoms. requirement 2: the radiation-sensitive acid generating substance contains an onium salt having two or more iodine atoms. requirement 3: the partial structure represented by the formula (1) has an iodine atom, and the radiation-sensitive acid generating substance contains an onium salt having an iodine atom. In the formula (1), Y¹ represents a divalent group represented by formula (2-1) or formula (2-2).

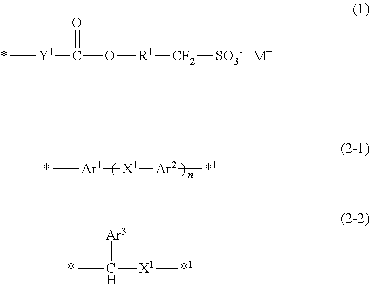

### CROSS-REFERENCE TO RELATED APPLICATIONS

The present application claims priority based on Japanese Patent Application No. 2024-018734, filed on Feb. 9, 2024, and Japanese Patent Application No. 2024-216987, filed on Dec. 11, 2024. The contents of these applications are incorporated herein by reference in their entirety.

### BACKGROUND OF THE DISCLOSURE

### Technical Field

The present disclosure relates to a radiation-sensitive composition, a method for forming a resist pattern, a polymer, and a compound.

### Discussion of the Background

In lithography technique used in manufacturing process of electronic devices such as semiconductor devices and liquid crystal devices, a radiation-sensitive composition is irradiated with far ultraviolet rays (such as ArF excimer laser), extreme ultraviolet rays (EUV), electron beams, etc. to generate an acid in an exposed portion, and a chemical reaction with this acid causes a difference in a dissolution rate in a developer liquid between the exposed portion and the unexposed portion to form a resist pattern on a substrate.

As the electronic device structures have been miniaturized, further miniaturization of the resist pattern in the lithography process has been required. With the requirement of further miniaturizing the resist pattern, various investigations have been made on improvement of resolution, shape of the resist pattern, etc. of the radiation-sensitive composition used for fine processing with lithography (for example, see Japanese Patent Laid-Open No. 2010-116550). Japanese Patent Laid-Open No. 2010-116550 discloses use of a polymer compound having a repeating unit having a sulfonium salt structure having a fluorine atom at an α-position of a sulfonic acid as a base resin of a radiation-sensitive composition.

### SUMMARY

One aspect according to the present disclosure provides a radiation-sensitive composition which contains a polymer having a partial structure represented by the following formula (1):

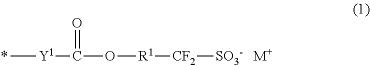

wherein Y¹ represents a divalent group represented by the following formula (2-1) or formula (2-2):

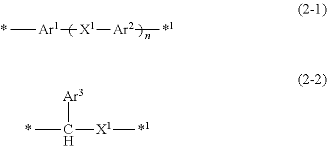

wherein Ar¹ represents a divalent aromatic ring group, Ar² represents a single bond or a divalent aromatic ring group, Ar³ represents a monovalent aromatic ring group, X¹ and X² each independently represent a divalent linking group, “n” represents 0 or 1, “*¹” represents an attachment point to the carbonyl group in the formula (1), and “*” represents an attachment point,
R¹ represents a divalent hydrocarbon group, M⁺ represents a sulfonium cation or an iodonium cation, and “*” represents an attachment point; and
a radiation-sensitive acid generating substance, wherein
the radiation-sensitive composition satisfies one or more of the following requirement 1, requirement 2, and requirement 3:
requirement 1: the partial structure represented by the formula (1) has two or more iodine atoms;
requirement 2: the radiation-sensitive acid generating substance contains an onium salt having two or more iodine atoms;
requirement 3: the partial structure represented by the formula (1) has an iodine atom, and the radiation-sensitive acid generating substance contains an onium salt having an iodine atom.

Another aspect according to the present disclosure provides a method for forming a resist pattern including: forming a resist film on a substrate by applying the above radiation-sensitive composition; exposing the resist film; and developing the exposed resist film.

Another aspect according to the present disclosure provides a polymer including a partial structure represented by the following formula (1):

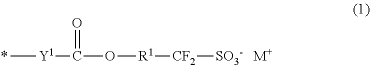

wherein Y¹ represents a divalent group represented by the following formula (2-1):

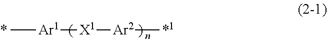

wherein Ar¹ represents a divalent aromatic ring group, Ar² represents a single bond or a divalent aromatic ring group, X¹ represents a divalent linking group, “n” represents 0 or 1, “*¹” represents an attachment point to the carbonyl group in the formula (1), and “*” represents an attachment point;
R¹ represents a divalent hydrocarbon group; M⁺ represents a sulfonium cation or an iodonium cation; and “*” represents an attachment point,
wherein the partial structure represented by the formula (1) has two or more iodine atoms.

Another aspect according to the present disclosure provides a polymer including a partial structure represented by the following formula (1):

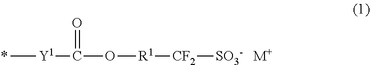

wherein Y¹ represents a divalent group represented by the following formula (2-2):

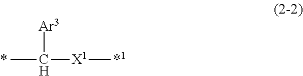

wherein Ar³ represents a monovalent aromatic ring group, X² represents a divalent linking group, “*¹” represents an attachment point to the carbonyl group in the formula (1), and “*” represents an attachment point,
R¹ represents a divalent hydrocarbon group; M⁺ represents a sulfonium cation or an iodonium cation, and “*” represents an attachment point.

Another aspect according to the present disclosure provides a compound represented by the following formula (3-1):

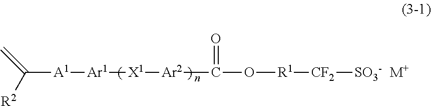

wherein R² represents a hydrogen atom, a fluorine atom, a methyl group, or a trifluoromethyl group; A¹ represents a single bond or *³—CO—O—; “*³” represents an attachment point to the carbon atom to which R² is bonded; Ar¹ represents a divalent aromatic ring group; Ar² represents a single bond or a divalent aromatic ring group; X¹ represents a divalent linking group; “n” represents 0 or 1; R¹ represents a divalent hydrocarbon group; and M⁺ represents a sulfonium cation or an iodonium cation, wherein the compound has two or more iodine atoms in the formula (3-1).

Another aspect according to the present disclosure provides a compound represented by the following formula (3-2):

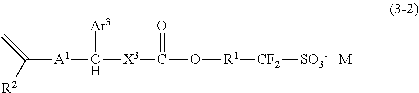

wherein R² represents a hydrogen atom, a fluorine atom, a methyl group, or a trifluoromethyl group; A¹ represents a single bond or *³—CO—O—; “*³” represents an attachment point to the carbon atom to which R² is bonded; Ar³ represents a monovalent aromatic ring group; X² represents a divalent linking group; R¹ represents a divalent hydrocarbon group; and M⁺ represents a sulfonium cation or an iodonium cation.

The present disclosure can provide a radiation-sensitive composition that has high sensitivity and excellent CUD performance and that can inhibit occurrence of development defects. The method for forming a resist pattern of the present disclosure uses the radiation-sensitive composition of the present disclosure, and thereby a resist pattern with good quality can be formed.

### DESCRIPTION OF THE EMBODIMENTS

The photolithography technique has achieved a finer pattern by utilizing radiation with a short wavelength, such as ArF excimer laser, and by using liquid-immersion lithography in which the exposure is performed in a state where a space between a lens of an exposure apparatus and the resist film is filled with a liquid medium. As next-generation technique, lithography using radiation with a shorter wavelength, such as electron beams, X-rays, and extreme ultraviolet rays (EUV), have been in progress. In these efforts toward the next-generation technique, it has been required to further improve radiation sensitivity, critical dimension uniformity (CDU: pattern size uniformity) performance, which is one of indexes indicating quality of the resist pattern, and an effect of inhibiting development defects.

Hereinafter, items relating to embodiments will be described in detail. Note that a numerical range described by using “to” herein has a meaning including values described before and after “to” as a lower limit and an upper limit.

Further, as used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

The “hydrocarbon group” herein has a meaning including a chain hydrocarbon group, an alicyclic hydrocarbon group, and an aromatic hydrocarbon group. The term “chain hydrocarbon group” means a linear hydrocarbon group and a branched hydrocarbon group that have no cyclic structure and that are constituted with only a chain structure. It is to be noted that the chain hydrocarbon group may be saturated or unsaturated. The term “alicyclic hydrocarbon group” means a hydrocarbon group that has only an alicyclic hydrocarbon structure as a cyclic structure and that has no aromatic cyclic structure. The alicyclic hydrocarbon group is not necessarily constituted with only the alicyclic hydrocarbon structure, and includes a group having a chain structure in a part thereof. The term “aromatic hydrocarbon group” means a hydrocarbon group having an aromatic cyclic structure as a cyclic structure. It is to be noted that the aromatic hydrocarbon group is not necessarily constituted with only the aromatic cyclic structure, and may have a chain structure or an alicyclic hydrocarbon structure in a part thereof. The term “aromatic ring group” refers to an n-valent group in which “n” (“n” represents an integer of 1 or more) hydrogen atoms are removed from a cyclic moiety of a substituted or unsubstituted aromatic ring. The term “aromatic heterocyclic group” refers to an n-valent group in which “n” (“n” represents an integer of 1 or more) hydrogen atoms are removed from a cyclic moiety of a substituted or unsubstituted aromatic heteroring. The term “organic group” means an atomic group in which any hydrogen atom is removed from a compound containing carbon (namely organic compound). The term “aromatic ring” has a meaning including an aromatic hydrocarbon ring and an aromatic heteroring.

The term “main chain” of a polymer refers to a moiety of “trunk” composed of a longest chain of atoms in the polymer. This “trunk” moiety is allowed to contain a cyclic structure. For example, an expression “having a specific structure in a main chain” refers to the specific structure constituting a part of the main chain of the polymer. The term “side chain” refers to a moiety branched from the “trunk” of the polymer. The term “structural unit” refers to a unit mainly constituting the main chain structure, and at least two or more of the units are contained in the main chain structure. The structural unit is typically a monomer unit. The term “(meth)acrylate” encompasses “acrylate” and “methacrylate”.

The description “substituted or unsubstituted p-valent hydrocarbon group”, wherein “p” represents an integer of 1 or more, encompasses a p-valent hydrocarbon group (namely, unsubstituted p-valent hydrocarbon group) and a group in which “p” hydrogen atoms are removed from a hydrocarbon structure moiety in a hydrocarbon group having a substituent. As an example of the substituted or unsubstituted p-valent hydrocarbon group, for example, an alkyl group and a fluoroalkyl group correspond to a case of p=1, and an alkanediyl group and a fluoroalkanediyl group correspond to a case of p=2. Among these, the fluoroalkyl group corresponds to “substituted monovalent hydrocarbon group”, and the fluoroalkanediyl group corresponds to “substituted divalent hydrocarbon group”. The same applies to other groups described with “substituted or unsubstituted”.

### <<Radiation-Sensitive Composition>>

The radiation-sensitive composition of the present disclosure (hereinafter, also referred to as “the present composition”) contains:
a polymer having a partial structure represented by the following formula (1):

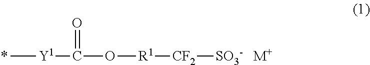

wherein Y¹ represents a divalent group represented by the following formula (2-1) or formula (2-2):

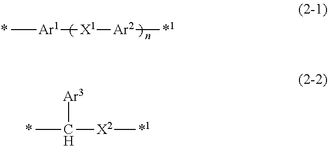

wherein Ar¹ represents a divalent aromatic ring group, Ar² represents a single bond or a divalent aromatic ring group, Ar³ represents a monovalent aromatic ring group, X¹ and X² each independently represent a divalent linking group, “n” represents 0 or 1, “*¹” represents an attachment point to a carbonyl group in the formula (1), and “*” represents an attachment point,
R¹ represents a divalent hydrocarbon group, M⁺ represents a sulfonium cation or an iodonium cation, and “*” represents an attachment point; and
a radiation-sensitive acid generating substance.

Hereinafter, the polymer having the partial structure represented by the formula (1) is also referred to as “polymer (P)”.

The present composition contains one or more components having an iodine atom. Specifically, the present composition satisfies one or more of the following requirement 1, requirement 2, and requirement 3.

Requirement 1: The partial structure represented by the formula (1) has two or more iodine atoms.

Requirement 2: The radiation-sensitive acid generating substance contains an onium salt having two or more iodine atoms.

Requirement 3: The partial structure represented by the formula (1) has an iodine atom, and the radiation-sensitive acid generating substance contains an onium salt having an iodine atom.

### <Partial Structure Represented by Formula (1)>

In the formula (1), examples of the divalent hydrocarbon group represented by R¹ include a linear or branched divalent saturated hydrocarbon group having 1 to 12 carbon atoms, a linear or branched divalent unsaturated hydrocarbon group having 2 to 12 carbon atoms, a divalent alicyclic hydrocarbon group having 3 to 12 carbon atoms, and a divalent aromatic hydrocarbon group having 6 to 12 carbon atoms.

Specific examples of the linear or branched divalent saturated hydrocarbon group having 1 to 12 carbon atoms include a methylene group, an ethylene group, a propane-1,3-diyl group, a propane-1,2-diyl group, a butane-1,4-diyl group, a butane-1,3-diyl group, a butane-2,3-diyl group, and a hexane-1,6-diyl group. Specific examples of the linear or branched divalent unsaturated hydrocarbon group having 2 to 12 carbon atoms include: alkenediyl groups such as an ethenediyl group, a propenediyl group, and a butenediyl group; and alkynediyl groups such as an ethynediyl group, a propynediyl group, and a butynediyl group.

Examples of the divalent alicyclic hydrocarbon group having 3 to 12 carbon atoms include a group having an alicyclic monocyclic hydrocarbon structure having 3 to 12 carbon atoms or an alicyclic polycyclic hydrocarbon structure having 6 to 12 carbon atoms as a cyclic structure. The alicyclic monocyclic hydrocarbon structure having 3 to 12 carbon atoms and the alicyclic polycyclic hydrocarbon structure having 6 to 12 carbon atoms may be any of saturated and unsaturated. The alicyclic polycyclic structure may be any of a bridged structure, a condensed cyclic structure, and a spiro-ring structure.

Examples of the ring in the alicyclic monocyclic hydrocarbon structure include a cyclopentane ring, a cyclohexane ring, a cycloheptane ring, a cyclooctane ring, a cyclopentene ring, a cyclohexene ring, a cycloheptene ring, a cyclooctene ring, and a cyclodecene ring. The alicyclic polycyclic hydrocarbon structure is preferably a bridged alicyclic saturated hydrocarbon structure or a condensed alicyclic saturated hydrocarbon structure, and examples thereof include a norbornane structure, a bicyclo[2.2.2]octane structure, and an adamantane structure.

Examples of the divalent aromatic hydrocarbon group having 6 to 12 carbon atoms include a group having an aromatic monocyclic hydrocarbon structure having 6 to 12 carbon atoms or an aromatic polycyclic hydrocarbon structure having 6 to 12 carbon atoms as a cyclic structure. Examples of the ring in the aromatic hydrocarbon group include a benzene ring and a naphthalene ring.

In terms of ability to more reduce an acidity of the sulfonic acid group (—SO₃⁻) and ability to more improve lithography performance (specifically, line width roughness (LWR) performance and CDU performance), R¹ preferably represents an alkanediyl group having 1 to 12 carbon atoms, more preferably represents an alkanediyl group having 1 to 8 carbon atoms, further preferably represents an alkanediyl group having 1 to 6 carbon atoms, and particularly preferably represents an alkanediyl group having 1 to 3 carbon atoms.

When Y¹ represents the group represented by the formula (2-1), the divalent aromatic ring group represented by Ar¹ or Ar² include a group in which two hydrogen atoms are removed from a cyclic moiety of a substituted or unsubstituted aromatic hydrocarbon ring and a group in which two hydrogen atoms are removed from a cyclic moiety of a substituted or unsubstituted aromatic heteroring. Among these, the divalent aromatic ring group is preferably a group in which two hydrogen atoms are removed from a cyclic moiety of a substituted or unsubstituted aromatic hydrocarbon ring. Examples of the aromatic hydrocarbon ring include a benzene ring, a naphthalene ring, an anthracene ring, and a phenanthrene ring. Among these, the aromatic hydrocarbon ring is preferably a benzene ring or a naphthalene ring, and more preferably a benzene ring. When the divalent aromatic ring group represented by Ar¹ or Ar² has a substituent in the cyclic moiety, examples of the substituent include an alkyl group having 1 to 3 carbon atoms, an alkoxy group having 1 to 3 carbon atoms, a halogen atom (such as a fluorine atom, a chlorine atom, a bromine atom, and an iodine atom), an acetyl group, a cyano group, and a nitro group.

When Y¹ represents the group represented by the formula (2-2), examples of the monovalent aromatic ring group represented by Ar³ include a group in which one hydrogen atom is removed from a cyclic moiety of a substituted or unsubstituted aromatic hydrocarbon ring and a group in which one hydrogen atom is removed from a cyclic moiety of a substituted or unsubstituted aromatic heteroring. Among these, the monovalent aromatic ring group is preferably a group in which one hydrogen atom is removed from a cyclic moiety of a substituted or unsubstituted aromatic hydrocarbon ring. Specific examples and preferable examples of the aromatic hydrocarbon ring and specific examples of the substituent respectively include rings and groups same as in the description of the divalent aromatic ring group represented by Ar¹ or Ar².

Examples of the divalent linking group represented by X¹ or X² include —O—, —S—, —CO—, —COO—, —NH—, —CO—NH—, —SO₂—, —(CH₂)ᵣ—O—, —(CH₂)ᵣ—O—(CH₂)ₛ—, and an alkanediyl group having 1 to 3 carbon atoms (“r” and “s” each independently represent an integer of 1 to 3). “n” represents 0 or 1. A case where “n” represents 0 and the requirement 1 is satisfied is preferable in terms of ability to yield the radiation-sensitive composition having more excellent sensitivity and CDU performance. A case where “n” represents 1, Y¹ in the formula (1) represents the group represented by the formula (2-1), and the radiation-sensitive acid generating substance (specifically, an acid generator described later) contained in the present composition has an iodine atom is preferable in terms of ability to yield the radiation-sensitive composition that can form a resist pattern with more inhibited development defects.

M⁺ in the formula (1) represents a sulfonium cation or an iodonium cation. M⁺ preferably represents an arylsulfonium cation or an aryliodonium cation, and more preferably represents a triarylsulfonium cation or a diaryliodonium cation in terms of ability to increase sensitivity of the present composition. A case where M⁺ represents a diaryliodonium cation and the requirement 1 is satisfied is further preferable in terms of ability to increase sensitivity of the present composition and ability to yield the radiation-sensitive composition having more excellent CDU performance while inhibiting occurrence of development defects. Note that the arylsulfonium cation refers to a cation in which one or more substituted or unsubstituted aryl groups are bonded to S⁺, and the triarylsulfonium cation refers to a cation in which three substituted or unsubstituted aryl groups are bonded to S⁺. Similarly, the aryliodonium cation refers to a cation in which one or more substituted or unsubstituted aryl groups are bonded to I⁺, and the diaryliodonium cation refers to a cation in which two substituted or unsubstituted aryl groups are bonded to I⁺.

In terms of ability to more increase sensitivity of the present composition, the partial structure represented by the formula (1) (hereinafter, also referred to as “specific partial structure”) preferably has an iodine atom. The number of iodine atoms per the specific partial structure is one or more. From the viewpoint of achievement of both increase in sensitivity and increase in CDU performance of the present composition, the number of iodine atoms per the specific partial structure is more preferably two or more. The number of iodine atoms per the specific partial structure is preferably six or less, and more preferably four or less from the viewpoint of ease of synthesis.

When the specific partial structure has an iodine atom, the anion moiety in the formula (1) (specifically, Y¹ in the formula (1)) may have the iodine atom, or the cation moiety (namely M⁺) may have the iodine atom. In addition, both the anion moiety and the cation moiety in the formula (1) may have the iodine atom. In terms of ability to yield the radiation-sensitive composition having higher sensitivity, the iodine atom in the specific partial structure is preferably bonded to an aromatic ring. The aromatic ring to which the iodine atom is bonded is preferably an aromatic hydrocarbon ring, more preferably a benzene ring and a naphthalene ring, and particularly preferably a benzene ring.

When the anion moiety in the specific partial structure has a plurality of the iodine atoms, at least a part of the plurality of the iodine atoms is preferably bonded to the aromatic ring, and all thereof are more preferably bonded to the aromatic ring. Each of the iodine atoms in the anion moiety in the specific partial structure may be bonded to one and the same aromatic ring or may be bonded to different aromatic rings. Similarly, when the cation moiety in the specific partial structure has a plurality of the iodine atoms, at least a part of the plurality of the iodine atoms is preferably bonded to the aromatic ring, and all thereof are more preferably bonded to the aromatic ring. Each of the iodine atoms in the cation moiety in the specific partial structure may be bonded to one and the same aromatic ring or may be bonded to different aromatic rings.

When the anion moiety in the specific partial structure has the iodine atom, one or both of Ar¹ and Ar² in the formula (2-1) preferably have the iodine atom as a substituent, or Ar³ in the formula (2-2) preferably has the iodine atom as a substituent. When the anion moiety in the specific partial structure has the iodine atom, one or both of Ar¹ and Ar² preferably represent an iodinated phenylene group, and it is more preferable that Ar¹ represent a diiodophenylene group and the requirement 1 be satisfied, that Ar¹ represent a diiodophenylene group and the requirement 3 be satisfied, or that Ar² represent an iodophenylene group and the requirement 3 be satisfied. In Ar¹ and Ar², a substituting position of the iodo group in the iodophenylene group and the diiodophenylene group is not particularly limited. The phenylene skeleton constituting the iodophenylene group and the diiodophenylene group may be any of a 1,4-phenylene group, a 1,3-phenylene group, and a 1,2-phenylene group. When the cation moiety (namely M⁺) in the specific partial structure has the iodine atom, M⁺ may be the iodonium cation, or may be a sulfonium cation having an iodine atom. The iodonium cation may further have an iodo group. When the cation moiety (namely M⁺) in the specific partial structure has the iodine atom, M⁺ preferably has two or more iodine atoms, and more preferably represents an iodonium cation having one or more iodo groups (—I) or a sulfonium cation having two or more iodine atoms.

A case where the cation moiety in the specific partial structure has one or more iodine atoms is preferable in terms of ability to increase the effect of inhibiting development defects. A case where the anion moiety in the specific partial structure has one or more iodine atoms is preferable in terms of ability to yield the radiation-sensitive composition having more excellent CDU performance. Particularly, a case where the cation moiety in the specific partial structure has one or more iodine atoms and the anion moiety has one or more iodine atoms is preferable in terms of ability to yield the radiation-sensitive composition having more excellent CDU performance while inhibiting development defects.

### <Radiation-Sensitive Acid Generating Substance>

The radiation-sensitive acid generating substance is a substance to generate an acid by irradiation with radiation. Preferably used as the radiation-sensitive acid generating substance is an onium salt having an onium cation (preferably a radiation-sensitive onium cation) and an organic anion that is a conjugated base of an acid. The organic anion is typically an anion in which a proton is removed from an acid group in an organic acid. Such a radiation-sensitive acid generating substance generates an acid derived from the organic anion by: decomposing the radiation-sensitive onium cation by an action of the radiation to release the organic anion; and bonding the released organic anion and hydrogen abstracted from a component contained in the present composition (for example, the radiation-sensitive acid generating substance itself or a solvent).

The radiation-sensitive acid generating substance may be a so-called radiation-sensitive acid generator, or may be an acid-diffusion inhibitor. The present composition may contain both the acid generator and the acid-diffusion inhibitor as the radiation-sensitive acid generating substance. Here, the acid generator is a substance to generate a strong acid in the present composition with exposure, and the strong acid can release the acid-dissociable group in the component therefrom in the radiation-sensitive composition. The acid-diffusion inhibitor is a substance that can inhibit diffusion of the acid in the resist film, and the acid is derived from the acid generator and generated by exposure to inhibit a chemical reaction by the acid in the unexposed portion. The radiation-sensitive acid generating substance is classified into the acid generator or the acid-diffusion inhibitor according to strength of the acid relative to the components in the present composition (specifically, the polymer (P) and another radiation-sensitive acid generating substance in a case of containing two or more radiation-sensitive acid generating substances). The degree of acidity can be evaluated with an acid dissociation constant (pKa). For example, an acid dissociation constant of the acid generated from the acid-diffusion inhibitor is typically −3 or more, preferably −1<pKa 7, and more preferably 0 pKa 5.

The radiation-sensitive acid generating substance is a component different from the polymer (P). That is, the radiation-sensitive acid generating substance is a substance that does not have the partial structure represented by the formula (1). In terms of easy regulation of sensitivity etc. of the present composition and in terms of high degree of freedom of selection of the radiation-sensitive acid generating substance to be blended in the present composition, a so-called low-molecular compound (also referred to as “non-polymer”) having no molecular weight distribution can be preferably used as the radiation-sensitive acid generating substance. The molecular weight of the radiation-sensitive acid generating substance is preferably 1,000 or less, more preferably 800 or less, and further preferably 600 or less.

In terms of ability to increase sensitivity of the present composition, the present composition preferably contains an onium salt in which one or both of the onium cation and the organic anion constituting the radiation-sensitive acid generating substance have an iodine atom (hereinafter, also referred to as “iodine-containing onium salt”). The number of the iodine atom per molecule of the iodine-containing onium salt may be one or more. From the viewpoint of achievement of both increase in sensitivity and increase in CDU performance of the present composition, the number of iodine atoms in the iodine-containing onium salt is more preferably two or more. The number of iodine atoms in the iodine-containing onium salt is preferably ten or less, and more preferably eight or less with considering balance between sensitivity and CDU performance and ease of synthesis.

The bonding position of the iodine atom in the iodine-containing onium salt is not particularly limited. In terms of ability to yield the radiation-sensitive composition having higher sensitivity, the iodine-containing onium salt preferably has a structure in which the iodine atom is bonded to an aromatic ring. The aromatic ring to which the iodine atom is bonded is preferably an aromatic hydrocarbon ring, more preferably a benzene ring and a naphthalene ring, and further preferably a benzene ring.

When the iodine-containing onium salt has a plurality of the iodine atoms, at least a part of the plurality of the iodine atoms is preferably bonded to the aromatic ring, and all thereof are more preferably bonded to the aromatic ring. Each of the iodine atoms in the iodine-containing onium salt may be bonded to one and the same aromatic ring or may be bonded to different aromatic rings in the iodine-containing onium salt.

The iodine-containing onium salt may be any as long as at least one of the onium cation and the organic anion has the iodine atom. Therefore, the iodine-containing onium salt may be an onium salt in which the onium cation has the iodine atom and the organic anion has no iodine atom, or may be an onium salt in which the organic anion has the iodine atom and the onium cation has no iodine atom. Alternatively, the iodine-containing onium salt may be an onium salt in which both the onium cation and the organic anion have the iodine atom. When the onium cation has the iodine atom, this onium cation may be an iodonium cation, or may be a sulfonium cation having the iodine atom. The iodonium cation may further have an iodo group.

A case where the organic anion in the iodine-containing onium salt has one or more iodine atoms is preferable in terms of ability to increase the effect of inhibiting occurrence of development defects. A case where the organic anion has an aromatic ring substituted with two or more iodo groups or a case where the organic anion has an aromatic ring substituted with a fluoro group or a trifluoromethyl group is preferable in terms of further increase in CDU performance.

The present composition may satisfy one or more of the requirement 1, the requirement 2, and the requirement 3. In terms of easy regulation of sensitivity, at least one or both of the requirement 1 and the requirement 2 are preferably satisfied. In term of ability to sufficiently yield the effect of inhibiting development defects while increasing sensitivity, at least the requirement 1 is more preferably satisfied.

A total number of the number of the iodine atoms per the specific partial structure and the number of the iodine atoms in one molecule of the iodine-containing onium salt (hereinafter, also referred to as “total number E”) may be two or more. From the viewpoint of achievement of solubility of the present composition and ease of synthesis of the polymer and the radiation-sensitive acid generating substance, the total number E is preferably 2 to 20, more preferably 2 to 15, further preferably 2 to 12, and particularly preferably 3 to 12.

### [As for Specific Aspect of Each Component in the Present Composition]

Next, the contained components and optionally blended components in the present composition will be further specifically described. The components contained in the present composition may be used with one type, or two or more types may be used in combination unless otherwise particularly mentioned.

### <Polymer (P)>

The polymer (P) may have the specific partial structure on the side chain, or on one terminal or both terminals of the polymer. In terms of ability to form the resist pattern with more reduced development defects, the polymer (P) preferably has a structural unit having the specific partial structure (hereinafter, also referred to as “structural unit (I)”).

### (Structural Unit (I))

The structural unit (I) is preferably a structural unit derived from an onium salt having the partial structure represented by the formula (1) and a polymerizable unsaturated bond. Preferable specific examples of the structural unit (I) include structural units represented by the following formula (3).

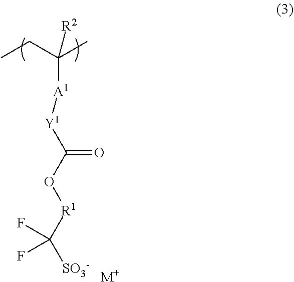

In the formula (3), R² represents a hydrogen atom, a fluorine atom, a methyl group, or a trifluoromethyl group. A¹ represents a single bond or *²—CO—O— (“*²” represents an attachment point to a main chain). Y¹, R¹, and M⁺ are as defined in the formula (1).

In the formula (3), R² preferably represents a hydrogen atom or a methyl group from the viewpoint of copolymerization properties. Specific examples and preferable examples of A¹, Y¹, and R¹ are as described in the formula (1).

Preferable specific examples of M⁺ include cations represented by the following formula (4), cations represented by the following formula (5), and cations represented by the following formula (6).

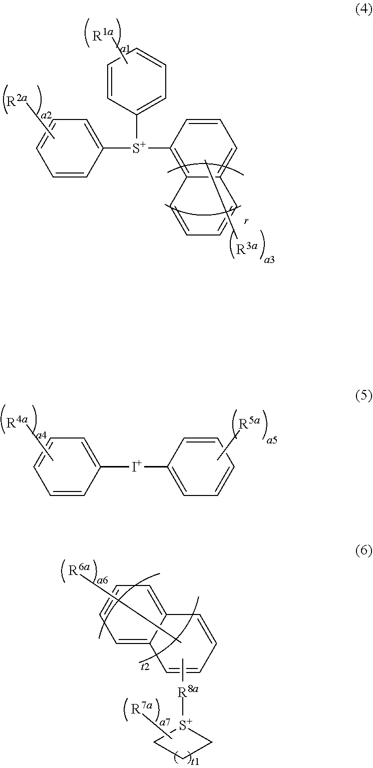

In the formula (4), R^{1a} and R^{2a} each independently represent a monovalent substituent, or represent a single bond or a divalent group linking a ring to which R^{1a} and R^{2a} are bonded by combining R^{1a} and R^{2a}. R^{3a} represents a monovalent substituent. a1 and a2 each independently represent an integer of 0 to 5. a3 represents an integer of 0 to (2*r+5). “r” represents 0 or 1.

In the formula (5), R^{4a} and R^{5a} each independently represent a monovalent substituent. a4 and a5 each independently represent an integer of 0 to 5.

In the formula (6), a6 represents an integer of 0 to 7. When a6 represents 1, R^{6a} represents a monovalent organic group having 1 to 20 carbon atoms, a hydroxy group, a nitro group, or a halogen group. When a6 represents 2 or more, a plurality of R^{6a} are the same or different, and represent a monovalent organic group having 1 to 20 carbon atoms, a hydroxy group, a nitro group, or a halogen group, or represent a cyclic structure having the number of ring member of 4 to 20 constituted by combining two of the plurality of R^{6a} together with the carbon atom to which the plurality of R^{6a} are bonded. a7 represents an integer of 0 to 6. When a7 represents 1, R^{7a} represents a monovalent organic group having 1 to 20 carbon atoms, a hydroxy group, a nitro group, or a halogen group. When a7 represents 2 or more, a plurality of R^{7a} are the same or different, and represent a monovalent organic group having 1 to 20 carbon atoms, a hydroxy group, a nitro group, or a halogen group, or represent a cyclic structure having the number of ring member of 3 to 20 constituted by combining two of the plurality of R^{7a} together with the carbon atom to which the plurality of R^{7a} are bonded. t1 represents an integer of 0 to 3. R^{8a} represents a single bond or a divalent organic group having 1 to 20 carbon atoms. t2 represents 0 or 1.

In the formula (4) and the formula (5), examples of the monovalent substituent represented by Ria, R^{2a}, R^{3a}, R^{4a}, and R^{5a} (hereinafter, referred to as “R^{1a} to R^{5a}”) include a fluorine atom, a chlorine atom, a bromine atom, an iodine atom, a substituted or unsubstituted alkyl group, a substituted or unsubstituted alkoxy group, a substituted or unsubstituted cycloalkyl group, a substituted or unsubstituted cycloalkyloxy group, an ester group, an alkylsulfonyl group, a cycloalkylsulfonyl group, a hydroxy group, a carboxy group, a cyano group, and a nitro group.

The alkyl group represented by R^{1a} to R^{5a} may be linear or branched. This alkyl group preferably has 1 to 10 carbon atoms. Among these, the alkyl group represented by R^{1a} to R^{5a} is preferably an alkyl group having 1 to 5 carbon atoms, and more preferably a methyl group, an ethyl group, an n-butyl group, or a t-butyl group. Specific examples of a case where R^{1a} to R^{5a} represents the alkoxy group include groups having the alkyl group exemplified above at an alkyl group moiety constituting the alkoxy group. This alkoxy group is preferably a methoxy group, an ethoxy group, an n-propoxy group, or an n-butoxy group.

The cycloalkyl group represented by R^{1a} to R^{5a} may be monocyclic or polycyclic. Among these, examples of the monocyclic cycloalkyl group include a cyclopropyl group, a cyclobutyl group, a cyclopentyl group, a cyclohexyl group, and a cyclooctyl group. Examples of the polycyclic cycloalkyl group include a norbornyl group, an adamantyl group, a tricyclodecyl group, and a tetracyclododecyl group. Specific examples of a case where R^{1a} to R^{5a} represent the cycloalkyloxy group include groups having the cycloalkyl group exemplified above at a cycloalkyl group moiety constituting the cycloalkyloxy group. The cycloalkyloxy group represented by R^{1a} to R^{5a} is preferably a cyclopentyloxy group or a cyclohexyloxy group.

When R^{1a} to R^{5a} have a substituent, examples of the substituent include a fluorine atom, a chlorine atom, a bromine atom, an iodine atom, a hydroxy group, a carboxy group, a cyano group, a nitro group, and an alkoxy group having 1 to 5 carbon atoms.

When R^{1a} to R^{5a} represent the ester group (—COOR), examples of a hydrocarbon moiety (R) in the ester group include the substituted or unsubstituted alkyl group or the substituted or unsubstituted cycloalkyl group exemplified above. Among these, R^{1a} to R^{5a} representing the ester group are preferably a methoxycarbonyl group, an ethoxycarbonyl group, or an n-butoxycarbonyl group. When R^{1a} to R^{5a} represent the alkylsulfonyl group, examples of an alkyl group moiety constituting the alkylsulfonyl group include the substituted or unsubstituted alkyl group exemplified above. When R^{1a} to R^{5a} represent the cycloalkylsulfonyl group, examples of an alkyl group moiety constituting this cycloalkylsulfonyl group include the substituted or unsubstituted cycloalkyl group exemplified above.

When R^{1a} and R^{2a} represent the divalent group linking a ring to which R^{1a} and R^{2a} are bonded by combining R^{1a} and R^{2a}, examples of this divalent group include —COO—, —OCO—, —CO—, —O—, —SO—, —SO₂—, —S—, an alkanediyl group having 1 to 3 carbon atoms, an alkanediyl group having 2 or 3 carbon atoms, and a group having —O—, —S—, —COO—, —OCO—, —CO—, —SO—, or —SO₂-between a carbon-carbon bond in an ethylene group. Among these, R^{1a} and R^{2a} preferably represent a single bond linking the ring, or form —O— or —S—.
a1, a2, and a3 each preferably represent an integer of 0 to 2. In terms of ability to more increase sensitivity of the radiation-sensitive composition, a1, a2, and a3 preferably satisfy a1+a2+a3≥1, and at least one of Ria, R^{2a}, and R^{3a} present in the formula (4) preferably represents at least one selected from the group consisting of a fluorine atom, an iodine atom, and a trifluoromethyl group.
a4 and a5 each independently represent an integer of 0 to 2. In terms of ability to more increase sensitivity of the radiation-sensitive composition, a4 and a5 preferably satisfy a4+a5≥1, and at least one of R^{4a} and R^{5a} present in the formula (5) preferably represents at least one selected from the group consisting of a fluorine atom, an iodine atom, and a trifluoromethyl group.

In the formula (6), examples of the monovalent organic group having 1 to 20 carbon atoms represented by R^{6a} and R^{7a} include a substituted or unsubstituted monovalent hydrocarbon group having 1 to 20 carbon atoms, —OR^{k}, —COOR^{k}, —O—CO—R^{k}, —O—R^{kk}—COOR^{k}, —R^{kk}—CO—R^{k}, —OSO₂—R^{k}, or —SO₂—R^{k}. R^{k} represents a monovalent hydrocarbon group having 1 to 10 carbon atoms. R^{kk} represents a single bond or a divalent hydrocarbon group having 1 to 10 carbon atoms. In R^{6a} and R^{7a}, examples of the substituent that replaces a hydrogen atom in the hydrocarbon group include groups same as the groups exemplified as the substituent in the group represented by R^{1a} to R^{5a}.

Examples of the divalent organic group represented by R^{8a} include a group in which one hydrogen atom is removed from a monovalent organic group having 1 to 20 carbon atoms exemplified as R^{6a} and R^{7a}.

Among the above, R^{6a} and R^{7a} preferably represent a fluorine atom, an iodine atom, a linear or branched monovalent alkyl group, a monovalent fluoroalkyl group, a monovalent aromatic hydrocarbon group, —OSO₂—R^{k} or —SO₂—R^{k}. a6 preferably represent an integer of 0 to 2, and more preferably 0 or 1. a7 preferably represent an integer of 0 to 2, and more preferably 0 or 1. t2 preferably represents 0. t1 preferably represents 2 or 3.

Specific examples of M⁺ include cations represented by each of the following formulae. M⁺ is not limited thereto.

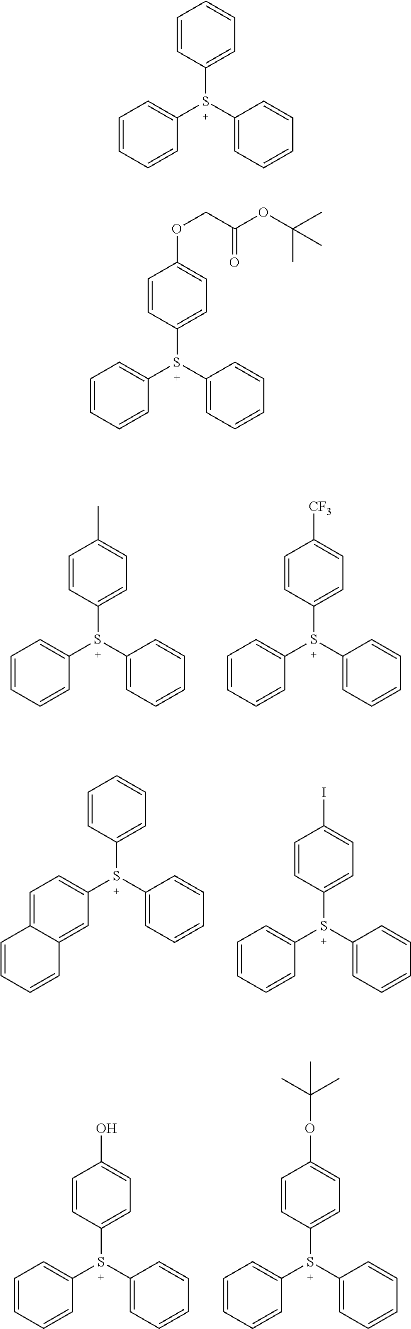

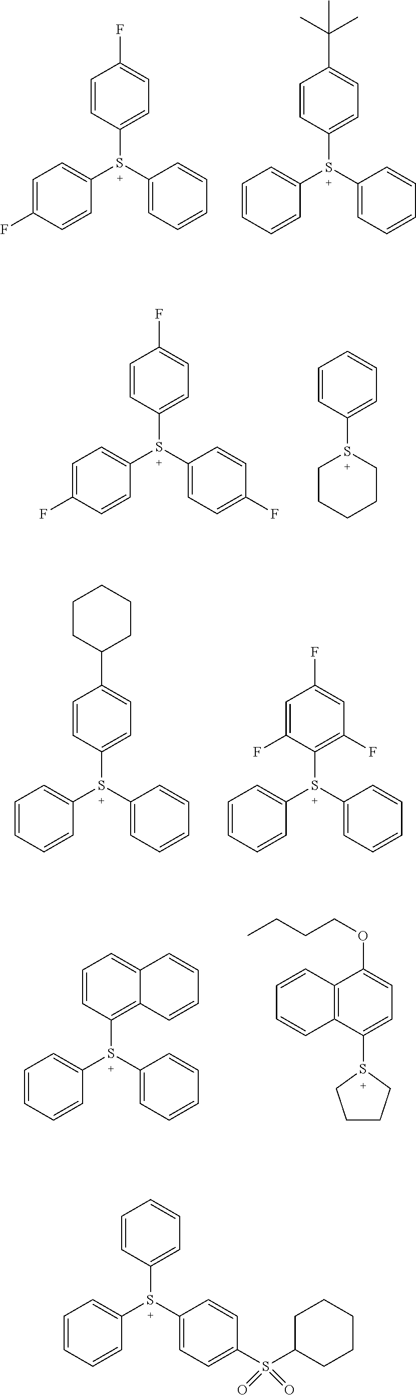

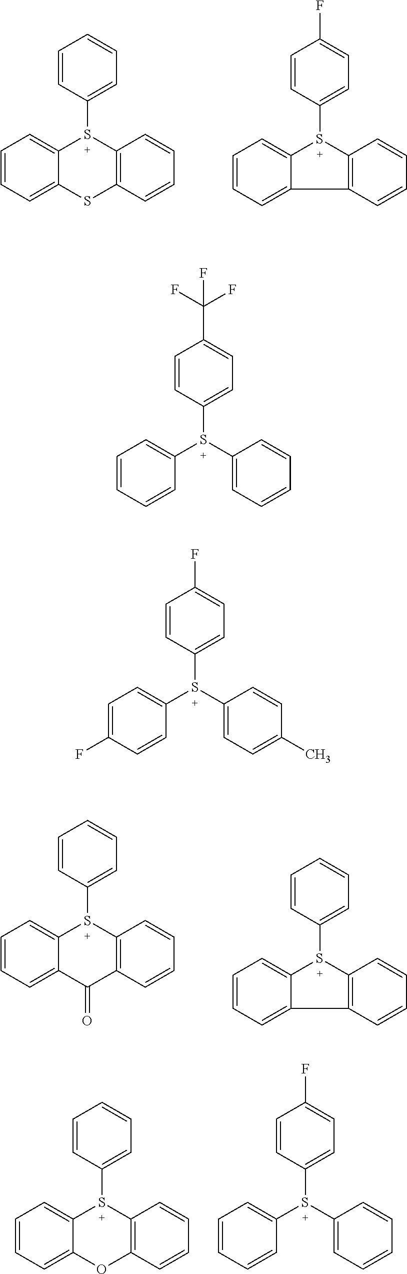

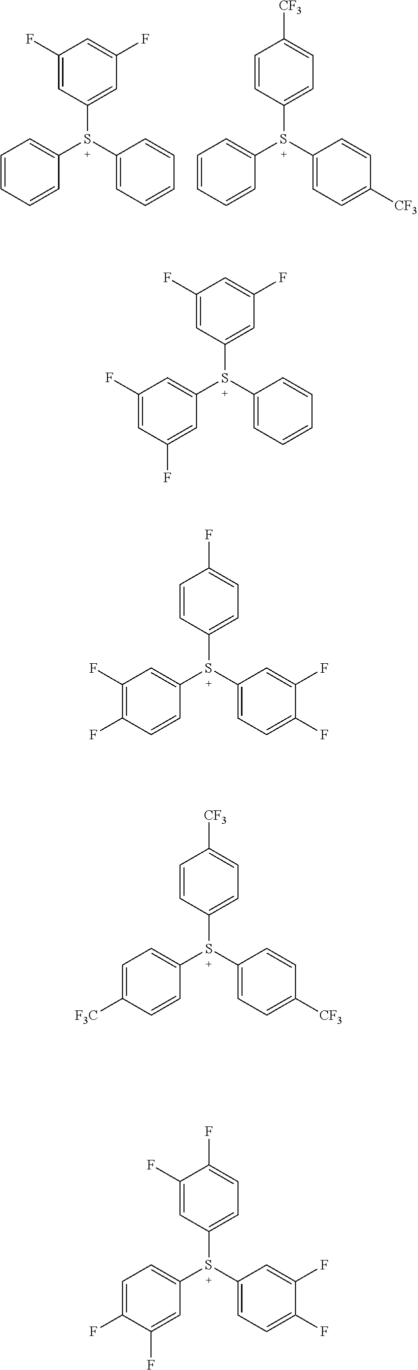

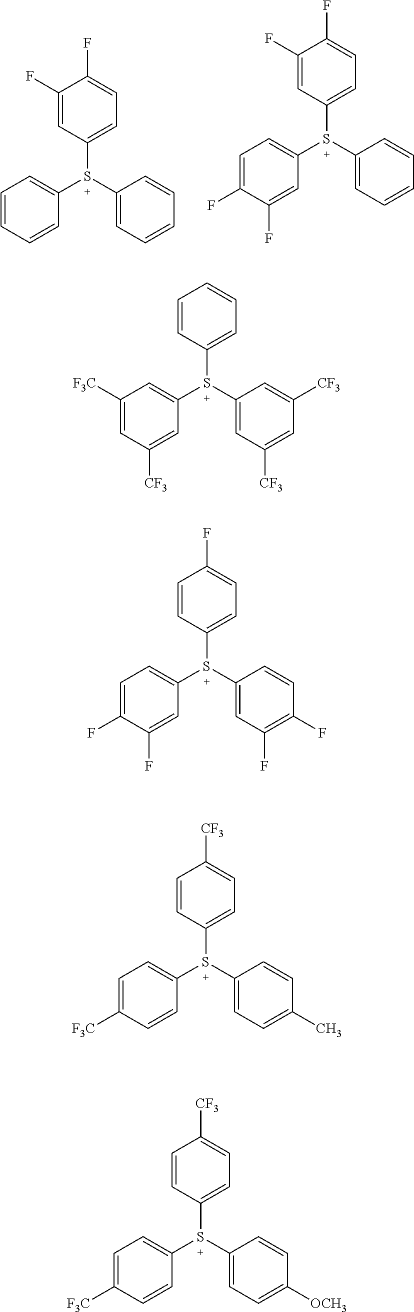

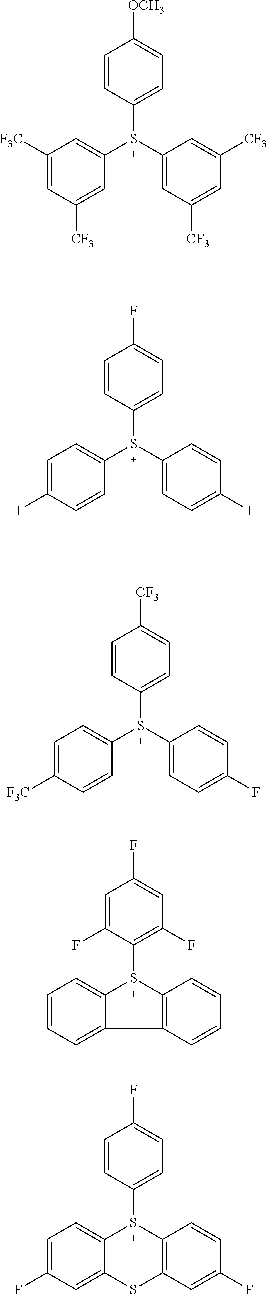

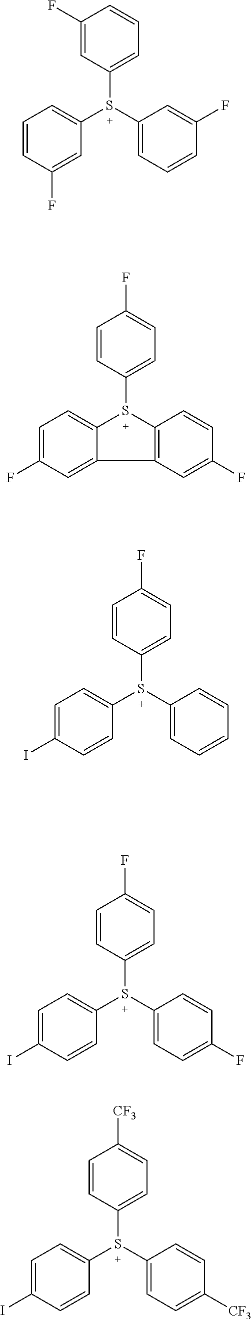

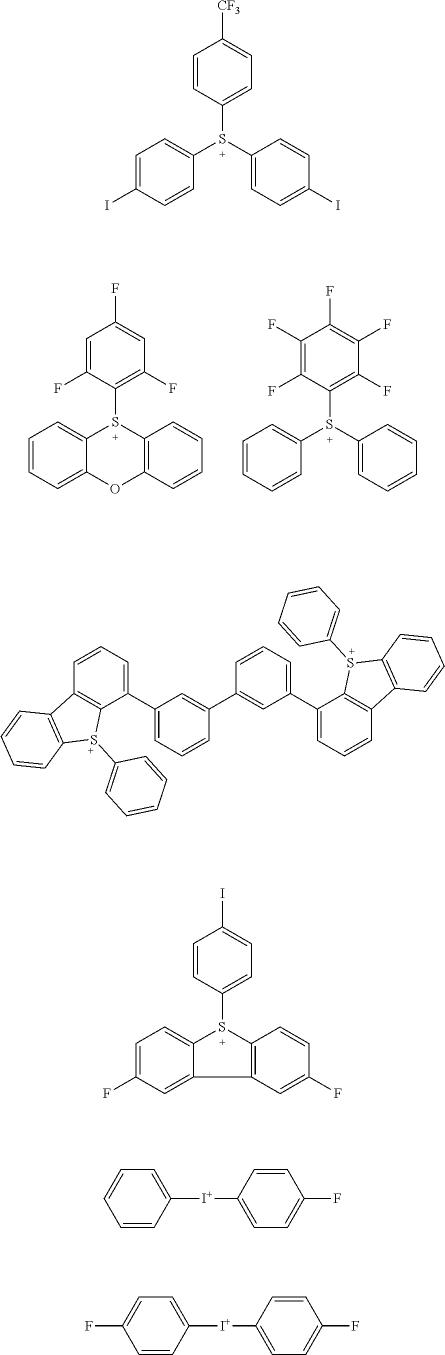

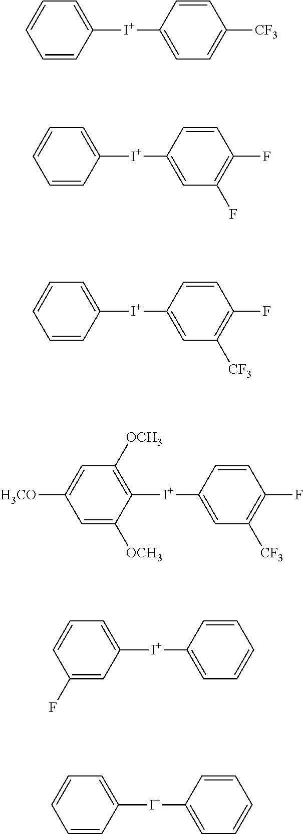 [SMILES: FC(F)(F)c1ccc([I+]c2ccccc2)cc1 ; Fc1ccc([I+]c2ccccc2)cc1F ; Fc1ccc([I+]c2ccccc2)cc1C(F)(F)F ; COc1cc(OC)c([I+]c2ccc(F)c(C(F)(F)F)c2)c(OC)c1 ; Fc1cccc([I+]c2ccccc2)c1 ; c1ccc([I+]c2ccccc2)cc1]

Specific examples of the structural unit (I) include structural units represented by the following formulae. The structural unit (I) is not limited to the following specific examples. In the formula, “M⁺” represents the monovalent cation, and specific examples thereof include the cation exemplified in the above.

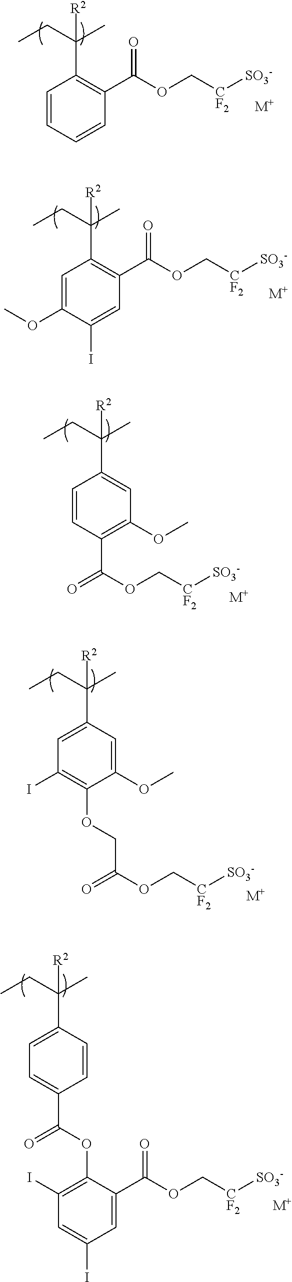

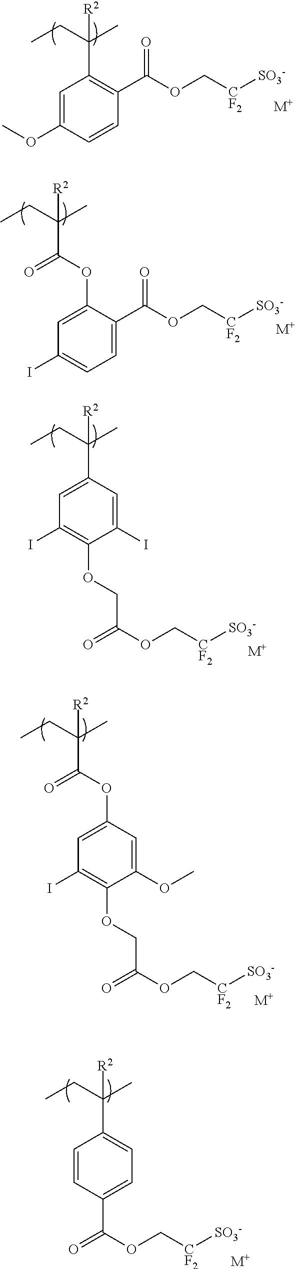

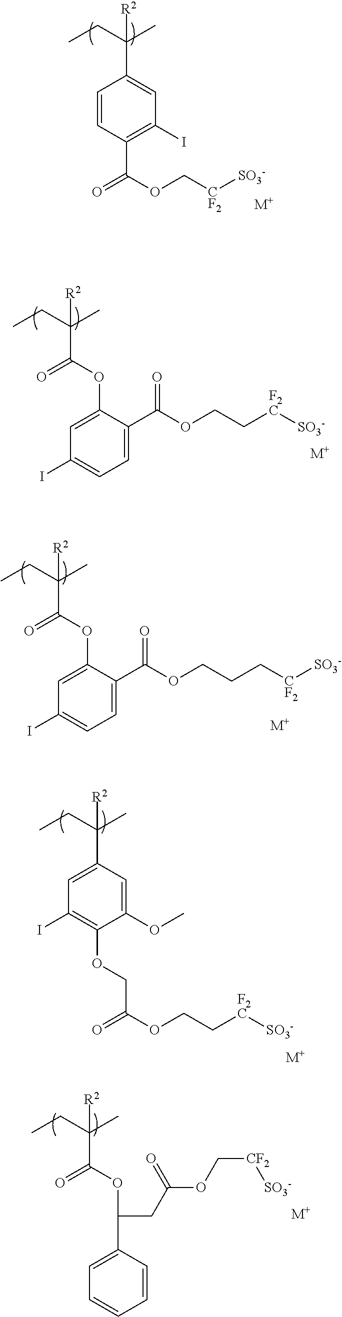

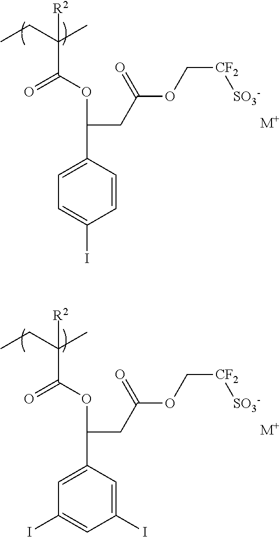

In the formulae, R² represents a hydrogen atom, a fluorine atom, a methyl group, or a trifluoromethyl group. M⁺ represents the sulfonium cation or the iodonium cation.

A content proportion of the structural unit (I) in the polymer (P) is preferably 1 mol % or more, more preferably 2 mol % or more, and further preferably 5 mol % or more relative to all the structural units constituting the polymer (P) from the viewpoint of increasing sensitivity and CDU performance, and sufficiently yielding the effect of improving development defect inhibition of the radiation-sensitive composition. The content proportion of the structural unit (I) is preferably 50 mol % or less, more preferably 40 mol % or less, and further preferably 30 mol % or less relative to all the structural units constituting the polymer (P) from the viewpoint of exhibiting good CDU performance of the radiation-sensitive composition. Setting the content proportion of the structural unit (I) to be within the above range can sufficiently yield the effect of reducing occurrence of development defects while retaining high sensitivity and good CDU performance.

### (Other Structural Units)

The polymer (P) may further have a structural unit different from the structural unit (I) (hereinafter, also referred to as “other structural unit”). Examples of the other structural unit include structural units (II) to (IV) described below.
Structural unit (II): a structural unit having an aromatic ring and a hydroxy group bonded to this aromatic ring
Structural unit (III): a structural unit having an acid-dissociable group
Structural unit (IV): a structural unit having a heterocyclic structure having a lactone structure, a cyclic carbonate structure, or a sultone structure, or a combination of two or more thereof
Structural unit (V): a structural unit having an alcoholic hydroxy group

Note that a structural unit having a hydroxy group bonded to an aromatic ring and an acid-dissociable group is classified to the structural unit (III) herein.

### Structural Unit (II)

The structural unit (II) is a structural unit having an aromatic ring and a hydroxy group bonded to this aromatic ring (except for the structural unit (I) and a structural unit having an acid-dissociable group). The polymer (P) further having the structural unit (II) is preferable in terms of ability to increase resolution of the present composition and in terms of the high effect of inhibiting elution of the unexposed portion into the developer liquid to sufficiently reduce development defects. Particularly in pattern formation using exposure with radiation having a wavelength of 50 nm or shorter, such as electron beams and EUV, the polymer having a hydroxy group bonded to an aromatic ring is preferably used.

Examples of the aromatic ring to which a hydroxy group is bonded include aromatic hydrocarbon rings such as a benzene ring, a naphthalene ring, an anthracene ring, and a phenanthrene ring. Among these, a benzene ring or a naphthalene ring is preferable, and a benzene ring is more preferable. The number of hydroxy groups bonded to an aromatic ring is not particularly limited. The number of hydroxy groups bonded to an aromatic ring is preferably one to three, and more preferably one or two. The position of the hydroxy group bonded to an aromatic ring is also not particularly limited. For example, when the structural unit (II) has a hydroxy group bonded to a benzene ring, the bonding position of the hydroxy group on the benzene ring in the structural unit (II) may be any of an ortho-position, a meta-position, and a para-position relative to another group.

On the aromatic ring to which the hydroxy group is bonded, a substituent different from the hydroxy group may be further bonded. Examples of this substituent include an alkyl group having 1 to 5 carbon atoms, an alkoxy group having 1 to 5 carbon atoms, a fluorine atom, a chlorine atom, a bromine atom, and an iodine atom.

Specific examples of the structural unit (II) include structural units represented by the following formula (7).

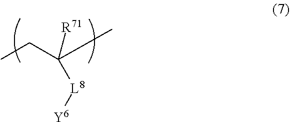

In the formula (7), R⁷?represents a hydrogen atom, a fluoro group, a methyl group, or a trifluoromethyl group. L⁸ represents a single bond, —O—, —CO—, *⁴—COO—, or *⁴—CONH—. “*⁴” represents an attachment point to a main chain. Y⁶ represents a monovalent group having the hydroxy group bonded to the aromatic ring.

In the formula (7), R⁷¹ preferably represents a hydrogen atom or a methyl group from the viewpoint of copolymerization properties of a monomer to yield the structural unit (II). L⁸ preferably represents a single bond or *⁴—COO—, and more preferably a single bond in terms of ability to more increase sensitivity of the present composition. Y⁶ preferably represents a group in which one hydrogen atom is removed from a cyclic moiety of the aromatic ring to which the hydroxy group is bonded (namely, a monovalent aromatic ring group substituted with the hydroxy group). The aromatic ring in Y⁶ may further have a substituent different from the hydroxy group. Specific examples of this substituent are as noted above.

Further specific examples of the structural unit (II) include structural units represented by the following formula.

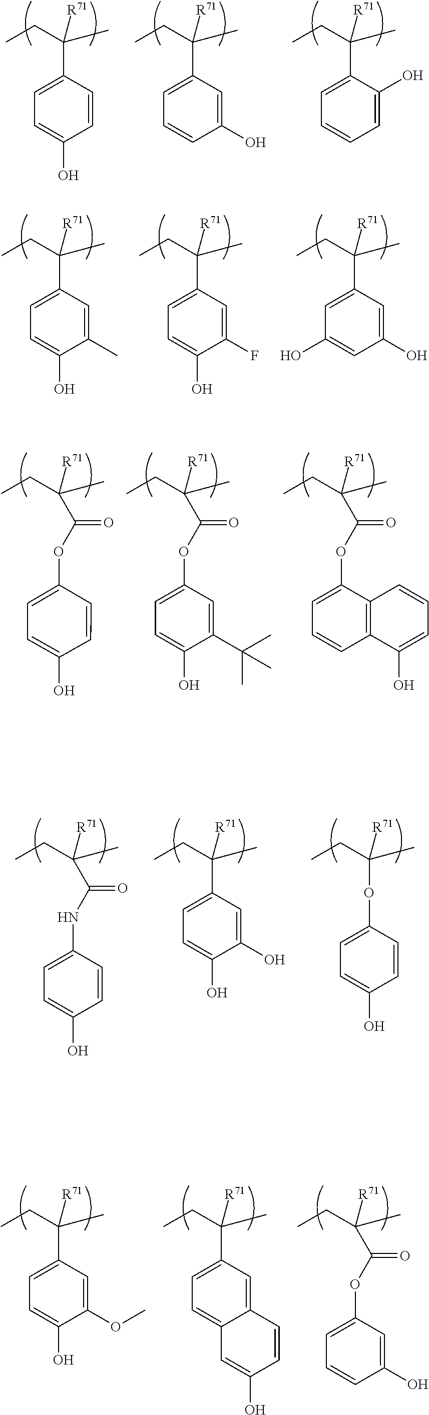

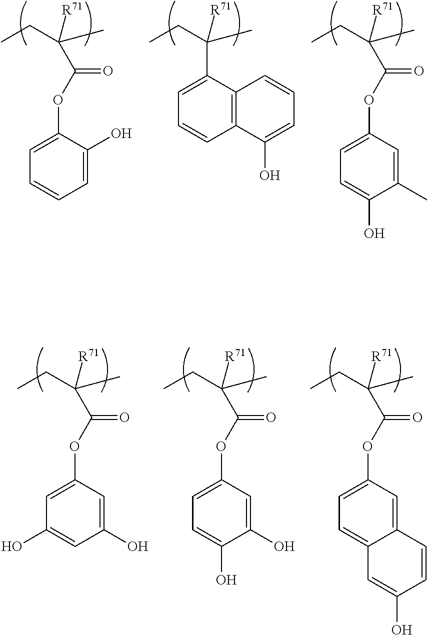

In the formula, R⁷¹ represents a hydrogen atom, a fluoro group, a methyl group, or a trifluoromethyl group.

When the polymer (P) has the structural unit (II), a content proportion of the structural unit (II) is preferably 1 mol % or more, more preferably 2 mol % or more, and further preferably 5 mol % or more relative to the total amount of the structural units constituting the polymer (P). The content proportion of the structural unit (II) is preferably 60 mol % or less, more preferably 50 mol % or less, and further preferably 45 mol % or less relative to the total amount of the structural units constituting the polymer (P). Setting the content proportion of the structural unit (II) to be within the above range is preferable in terms of ability to further improve resolution of the present composition.

### Structural Unit (III)

The acid-dissociable group in the structural unit (III) is a group that substitutes a hydrogen atom in an acid group such as a carboxy group and a hydroxy group and that is dissociated by an action of the acid. The polymer having the acid-dissociable group contained in the present composition dissociates the acid-dissociable group by an acid generated by exposure to generate the acid group, which can change solubility of the polymer component in the developer liquid. This can impart good lithography performance (specifically, LWR performance and critical dimension uniformity (CDU) performance) to the present composition, and can form the good resist pattern.

The structural unit (III) is not particularly limited as long as the structural unit has the acid-dissociable group. Examples of the structural unit (III) include a structural unit represented by the following formula (8-1) (hereinafter, also referred to as “structural unit (3A)”), a structural unit represented by the following formula (8-2) (hereinafter, also referred to as “structural unit (3B)”), and a structural unit represented by the following formula (8-3) (hereinafter, also referred to as “structural unit (3C)”).

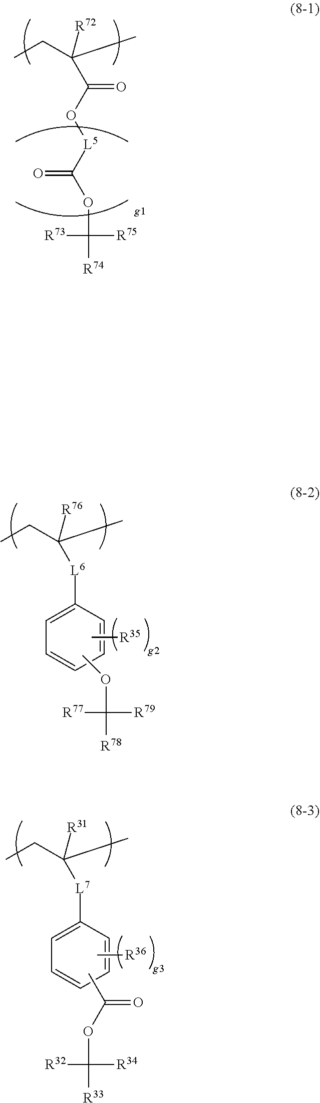

In the formula (8-1), R⁷² include a hydrogen atom, a fluorine atom, a methyl group, or a trifluoromethyl group. L⁵ represents a divalent chain organic group or an alicyclic hydrocarbon group. R⁷³ represents a hydrogen atom or a substituted or unsubstituted monovalent hydrocarbon group having 1 to 20 carbon atoms. R⁷⁴ and R⁷⁵ each independently represent a substituted or unsubstituted monovalent hydrocarbon group having 1 to 20 carbon atoms or a monovalent aromatic heterocyclic group, or represent an alicyclic hydrocarbon structure having 3 to 20 carbon atoms constituted by combining R⁷⁴ and R⁷⁵ together with the carbon atom to which R⁷⁴ and R⁷⁵ are bonded. When R⁷³ represents a hydrogen atom, any one or both of R⁷⁴ and R⁷⁵ each independently represent a substituted or unsubstituted monovalent unsaturated hydrocarbon group or a monovalent aromatic heterocyclic group, or an alicyclic unsaturated hydrocarbon structure having 3 to 20 carbon atoms constituted by combining R⁷⁴ and R⁷⁵ together with the carbon atom to which R⁷⁴ and R⁷⁵ are bonded. g1 represents 0 or 1.

In the formula (8-2), R⁷⁶ represents a hydrogen atom, a fluorine atom, a methyl group, or a trifluoromethyl group. L⁶ represents a single bond, —O—, —CO—, *⁵—COO—, or *⁵—CONH—. “*⁵” represents an attachment point bonded to a main chain. R⁷⁷, R⁷⁸, and R⁷⁹ each independently represent a hydrogen atom, a substituted or unsubstituted monovalent hydrocarbon group having 1 to 20 carbon atoms, or a substituted or unsubstituted monovalent oxyhydrocarbon group having 1 to 20 carbon atoms. R³⁵ represents a monovalent substituent. g2 represents an integer of 0 to 4.

In the formula (8-3), R³¹ represents a hydrogen atom, a fluorine atom, a methyl group, or a trifluoromethyl group. L⁷ represents a single bond, —O—, —CO—, *⁶—COO—, or *⁶—CONH—. “*⁶” represents an attachment point to a main chain. R³² represents a hydrogen atom, a substituted or unsubstituted monovalent hydrocarbon group having 1 to 20 carbon atoms, or a substituted or unsubstituted monovalent oxyhydrocarbon group having 1 to 20 carbon atoms. R³³ and R³⁴ each independently represent a hydrogen atom, a substituted or unsubstituted monovalent hydrocarbon group having 1 to 20 carbon atoms, or a substituted or unsubstituted monovalent oxyhydrocarbon group having 1 to 20 carbon atoms, or represent an alicyclic hydrocarbon structure having 3 to 20 carbon atoms constituted by combining R³³ and R³⁴ together with the carbon atom to which R³³ and R³⁴ are bonded. R³⁶ represents a monovalent substituent. g3 represents an integer of 0 to 4.

In the formula (8-1), R⁷² preferably represents a hydrogen atom or a methyl group, and more preferably a methyl group from the viewpoint of copolymerization properties of a monomer to yield the structural unit (3A). In the formula (8-2), R⁷⁶ preferably represents a hydrogen atom from the viewpoint of copolymerization properties of a monomer to yield the structural unit (3B). Similarly, R³¹ in the formula (8-3) preferably represents a hydrogen atom or a methyl group. L⁶ in the formula (8-2) or L⁷ in the formula (8-3) preferably represents a single bond, *⁶—COO—, or *⁶—CONH—.

Examples of the divalent chain organic group represented by L⁵ in the formula (8-1) include a linear or branched saturated hydrocarbon group having 1 to 20 carbon atoms and a divalent group having 2 to 20 carbon atoms in which a methylene group contained in a linear or branched saturated hydrocarbon group is replaced by a hetero-atom-containing group (for example, —O—, —S—, —CO—, —COO—, —NH—, —NHCO—, or —SO₂—). Specific examples and preferable examples of the divalent alicyclic hydrocarbon group represented by L⁵ include groups same as groups exemplified below as a monovalent alicyclic hydrocarbon group represented by R⁷³ to R⁷⁵, R⁷⁷ to R⁷⁹, or R³² to R³⁴. L⁵ preferably represents a chain organic group.

Examples of the monovalent hydrocarbon group having 1 to 20 carbon atoms represented by R⁷³ to R⁷⁵, R⁷⁷ to R⁷⁹, or R³² to R³⁴ include a monovalent chain hydrocarbon group having 1 to 20 carbon atoms, a monovalent alicyclic hydrocarbon group having 3 to 20 carbon atoms, and a monovalent aromatic hydrocarbon group having 6 to 20 carbon atoms.

Examples of the monovalent chain hydrocarbon group having 1 to 20 carbon atoms include: alkyl groups such as a methyl group, an ethyl group, an n-propyl group, an i-propyl group, an n-butyl group, and a t-butyl group; alkenyl groups such as an ethenyl group, a propenyl group, and a butenyl group; and alkynyl groups such as an ethynyl group, a propynyl group, and a butynyl group. Among these, the monovalent chain hydrocarbon group having 1 to 20 carbon atoms represented by R⁷³ to R⁷⁵, R⁷⁷ to R⁷⁹, or R³² to R³⁴ is preferably an alkyl group, and more preferably an alkyl group having 1 to 4 carbon atoms.

Examples of the monovalent alicyclic hydrocarbon group having 3 to 20 carbon atoms include: monovalent monocyclic alicyclic saturated hydrocarbon groups such as a cyclopentyl group, a cyclohexyl group, a methylcyclopentyl group, an ethylcyclopentyl group, a methylcyclohexyl group, and an ethylcyclohexyl group; monovalent monocyclic alicyclic unsaturated hydrocarbon groups such as a cyclopentenyl group, a cyclohexenyl group, a methylcyclopentenyl group, and a methylcyclohexenyl group; monovalent polycyclic alicyclic saturated hydrocarbon groups such as a norbornyl group, an adamantyl group, and a tricyclodecyl group; and monovalent polycyclic alicyclic unsaturated hydrocarbon groups such as a norbornenyl group, a tricyclodecenyl group, and an indanyl group.

Examples of the monovalent aromatic hydrocarbon group having 6 to 20 carbon atoms include: aryl groups such as a phenyl group, a tolyl group, a xylyl group, a mesityl group, a naphthyl group, a methylnaphthyl group, an anthryl group, a methylanthryl group, and an indenyl group; and aralkyl groups such as a benzyl group, a phenethyl group, a naphthylmethyl group, and an anthrylmethyl group.

Examples of the monovalent unsaturated hydrocarbon group represented by R⁷³ or R⁷⁴ include the aforementioned monocyclic or polycyclic alicyclic unsaturated hydrocarbon group and aromatic hydrocarbon group. Examples of the monovalent aromatic heterocyclic group include a furyl group and a thienyl group.

Examples of the alicyclic hydrocarbon structure having 3 to 20 carbon atoms constituted by combining R⁷⁴ and R⁷⁵ together with the carbon atom to which R⁷⁴ and R⁷⁵ are bonded and the alicyclic hydrocarbon structure having 3 to 20 carbon atoms constituted by combining R³³ and R³⁴ together with the carbon atom to which R³³ and R³⁴ are bonded include: monocyclic alicyclic saturated hydrocarbon structures such as a cyclopropane structure, a cyclobutane structure, a cyclopentane structure, a cyclohexane structure, a cycloheptane structure, and a cyclooctane structure; monocyclic alicyclic unsaturated hydrocarbon structures such as cyclopentene and cyclohexene; and polycyclic alicyclic hydrocarbon structures such as a norbornane structure, an adamantane structure, a tricyclodecane structure, and a tetracyclododecane structure.

Examples of the monovalent oxyhydrocarbon group having 1 to 20 carbon atoms represented by R⁷⁷ to R⁷⁹ or R³² to R³⁴ include a group having an oxygen atom on a terminal on the attachment point side of the group exemplified as the monovalent hydrocarbon group having 1 to 20 carbon atoms represented by R⁷³ to R⁷⁵, R⁷⁷ to R⁷⁹, and R³² to R³⁴. Among these, the monovalent oxyhydrocarbon group represented by R⁷⁷ to R⁷⁹ or R³² to R³⁴ is preferably an alkoxy group, a cycloalkoxy group, or a cycloalkylalkoxy group.

When the group represented by R⁷³ to R⁷⁵, R⁷⁷ to R⁷⁹, and R³² to R³⁴ has a substituent, examples of this substituent include a halogen atom (such as a fluorine atom, a chlorine atom, a bromine atom, and an iodine atom), a hydroxy group, and an alkoxy group having 1 to 3 carbon atoms. When R⁷⁴ and R⁷⁵ are combined to constitute the alicyclic hydrocarbon structure having 3 to 20 carbon atoms together with the carbon atom to which R⁷⁴ and R⁷⁵ are bonded or when R³³ and R³⁴ are combined to constitute the alicyclic hydrocarbon structure having 3 to 20 carbon atoms together with the carbon atom to which R³³ and R³⁴ are bonded, the substituent and the alkyl group exemplified above may be bonded to the ring.

Examples of the monovalent substituent represented by R³⁵ or R³⁶ include an alkyl group having 1 to 3 carbon atoms, an alkoxy group having 1 to 3 carbon atoms, a fluorine atom, a chlorine atom, a bromine atom, and an iodine atom. g2 and g3 preferably represent 0 to 2, and more preferably 0 or 1.

A case where at least a part of the structural unit (III) has at least one group selected from the group consisting of an iodine atom, a fluorine atom, and a trifluoromethyl group (hereinafter, also referred to as “specific group”) is preferable in terms of ability to more improve CDU performance while increasing sensitivity of the present composition. In the structural unit (III), the specific group is preferably bonded to an aromatic ring, and it is more preferable that the acid-dissociable group have an aromatic ring and the specific group be bonded to this aromatic ring. The number of the specific groups (when two or more types of the specific group are contained, a total number thereof) in the monomer to yield the structural unit (III) is preferably one to ten, and more preferably one to six.

Specific examples of the structural unit (3A) include structural units represented by the following formulae.

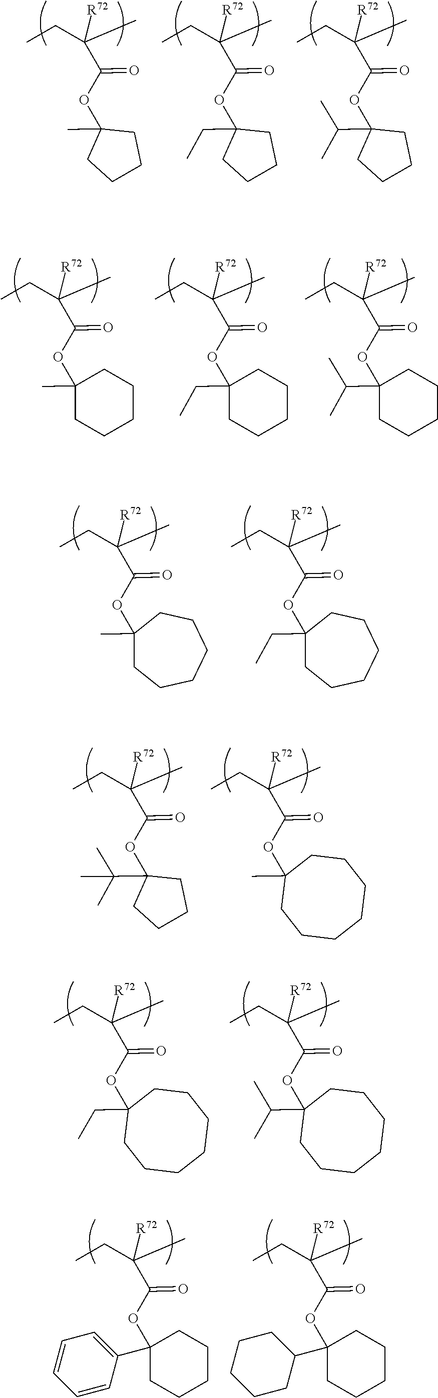

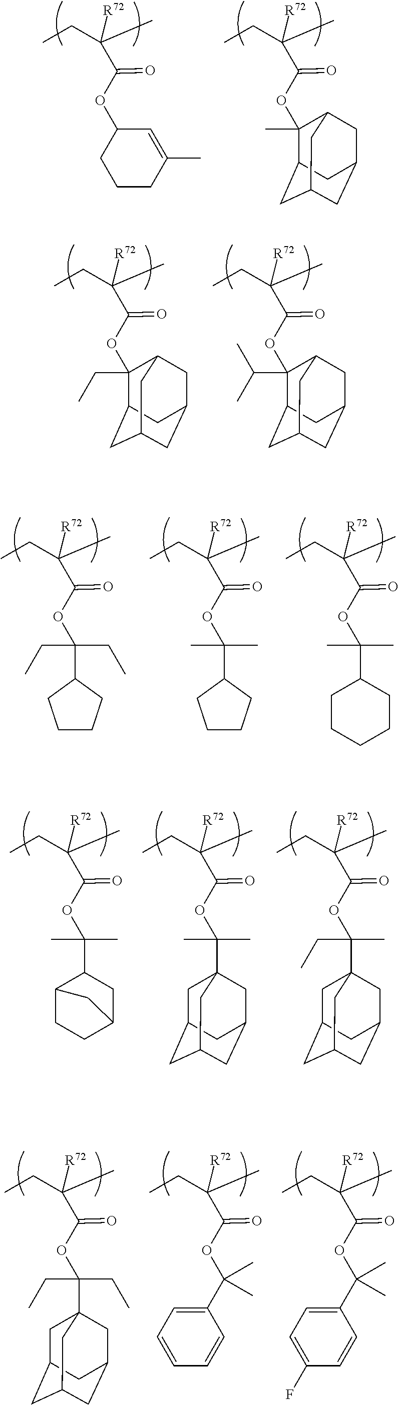

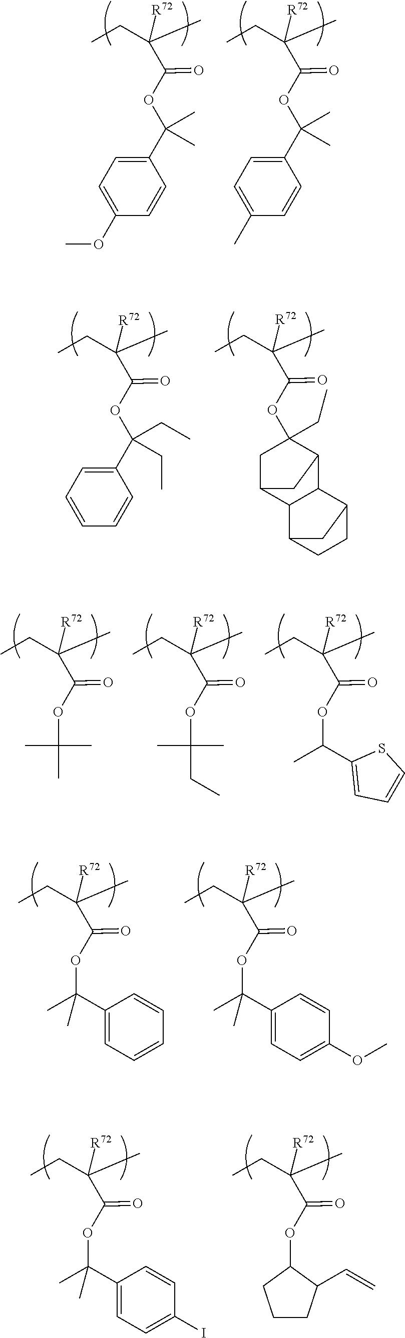

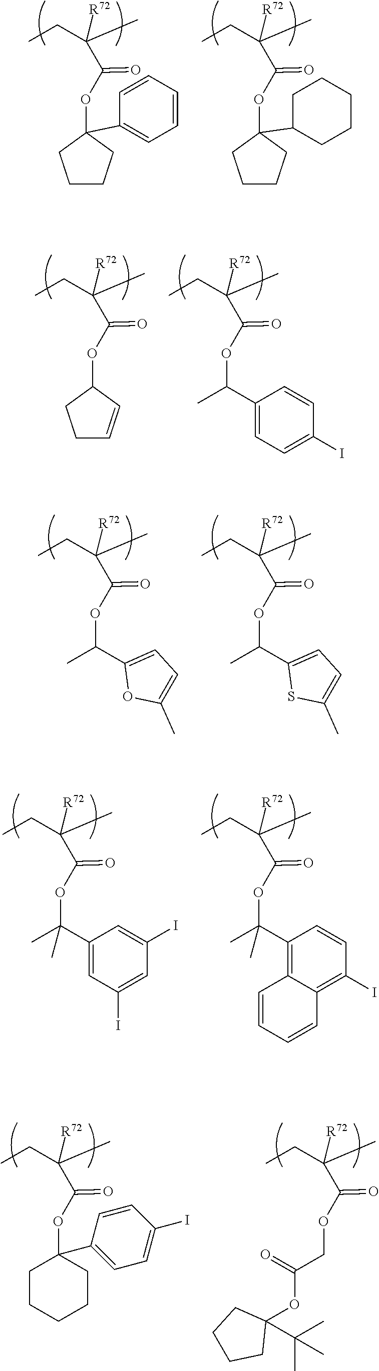

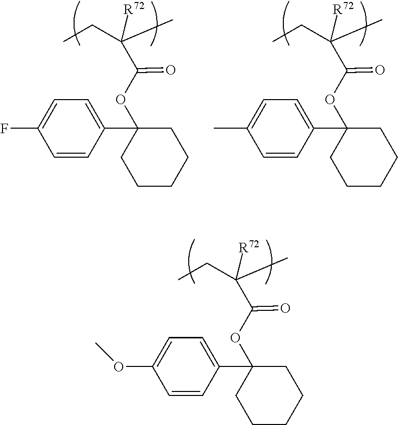

In the formulae, R⁷² represents a hydrogen atom, a fluorine atom, a methyl group, or a trifluoromethyl group.

Specific examples of the structural unit (3B) include structural units represented by the following formula.

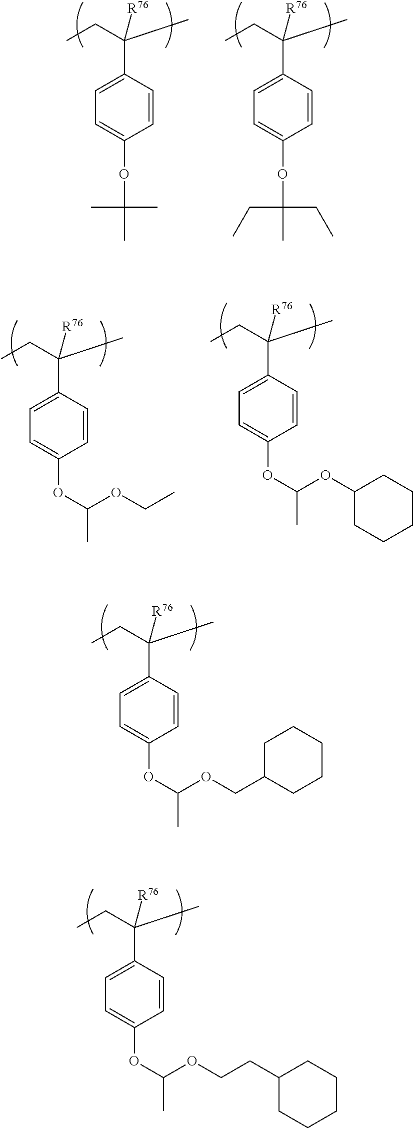

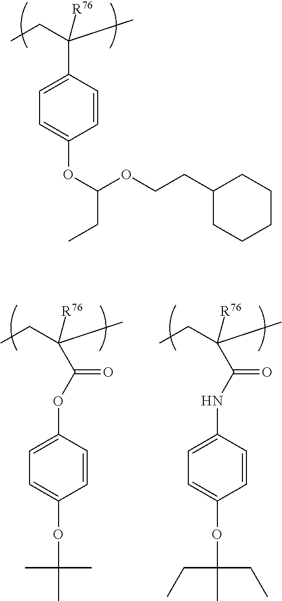

In the formula, R⁷⁶ represents a hydrogen atom, a fluorine atom, a methyl group, or a trifluoromethyl group.

Specific examples of the structural unit (3C) include structural units represented by the following formulae.

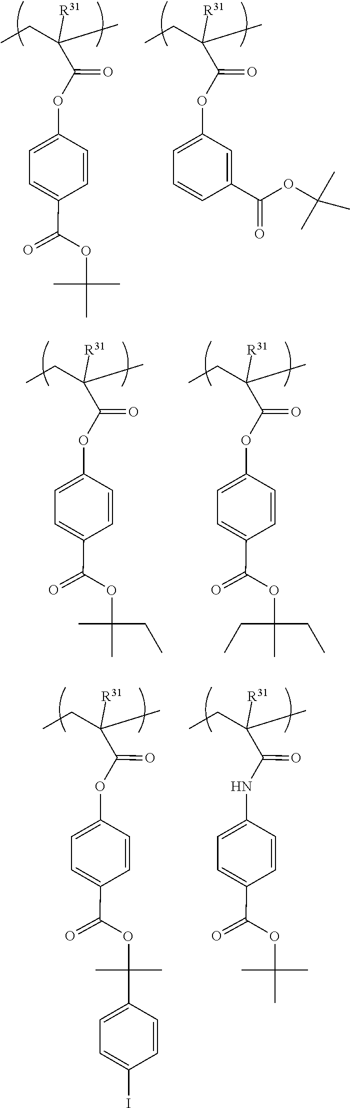

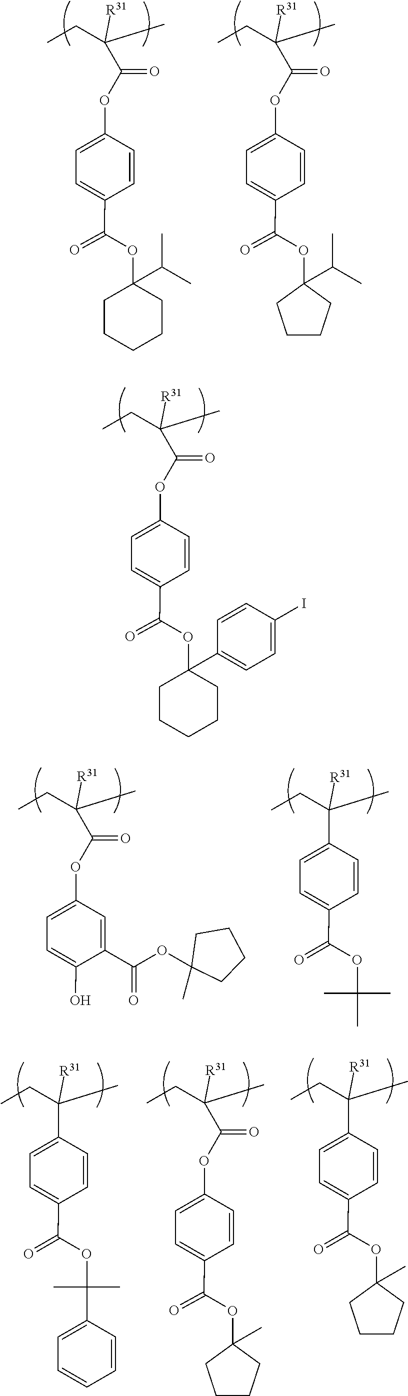

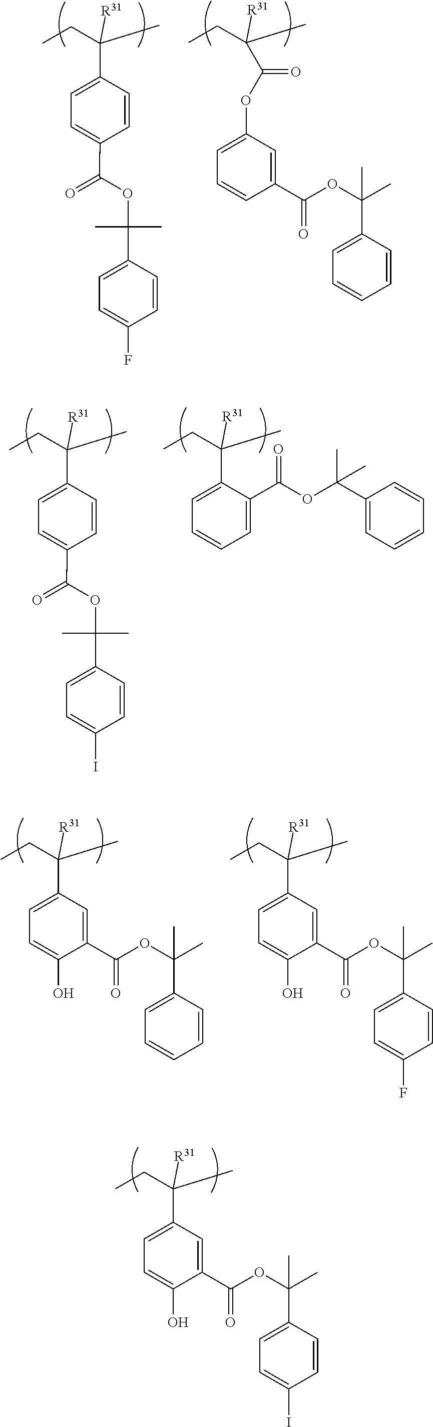

In the formula, R³¹ represents a hydrogen atom, a fluorine atom, a methyl group, or a trifluoromethyl group.

Other examples of the structural unit (III) include structural units derived from a compound in which two carboxy groups in an unsaturated dicarboxylic acid are protected (for example, di-tert-butyl maleate etc.).

In the polymer (P), a content proportion of the structural unit (III) is preferably 10 mol % or more, more preferably 30 mol % or more, further preferably 50 mol % or more, and further preferably 60 mol % or more relative to the total amount of the structural units constituting the polymer (P) from the viewpoint of more increasing sensitivity of the present composition. The content proportion of the structural unit (III) is preferably 85 mol % or less, more preferably 80 mol % or less, and further preferably 75 mol % relative to the total amount of the structural units constituting the polymer (P). Setting the content proportion of the structural unit (III) to be within the above range can more increase a difference in a dissolution rate of the exposed portion and the unexposed portion in the developer liquid to more improve the pattern shape of the resist film while retaining sensitivity of the present composition.

### Structural Unit (IV)

The structural unit (IV) is a structural unit having a cyclic structure having a lactone structure, a cyclic carbonate structure, or a sultone structure, or a combination of two or more thereof (excluding the structural unit (I) to the structural unit (III)).

Specific examples of the structural unit (IV) include structural units represented by the following formulae.

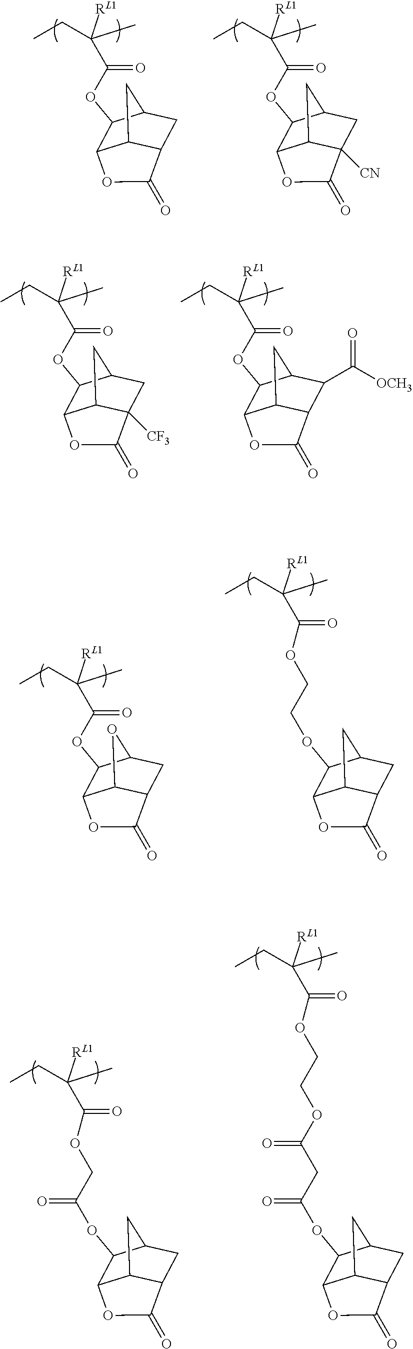

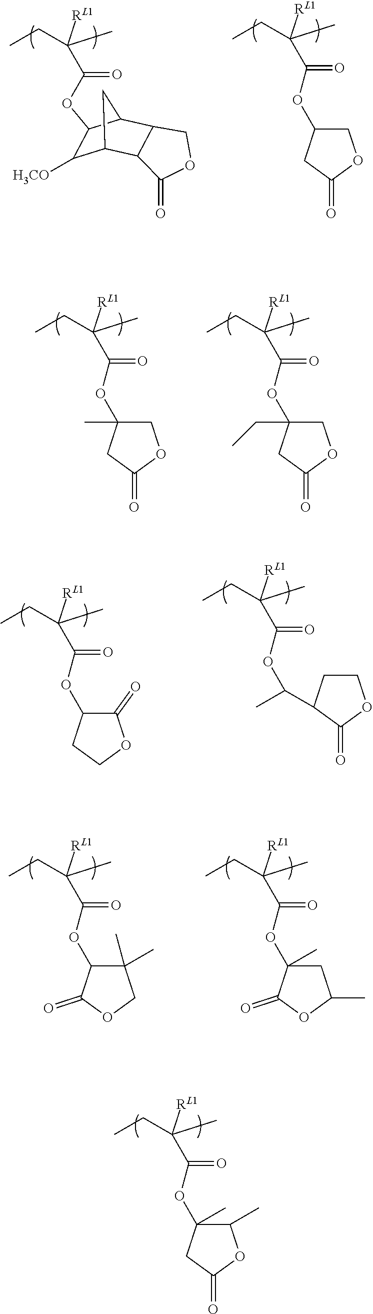

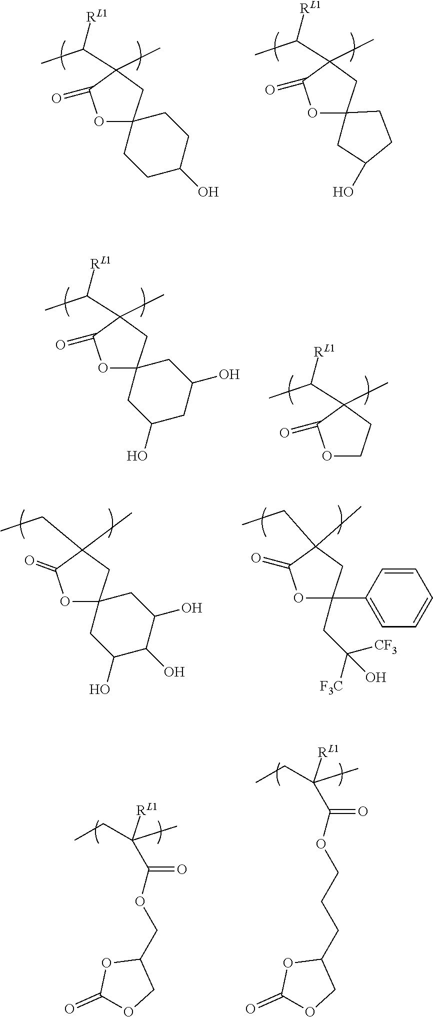

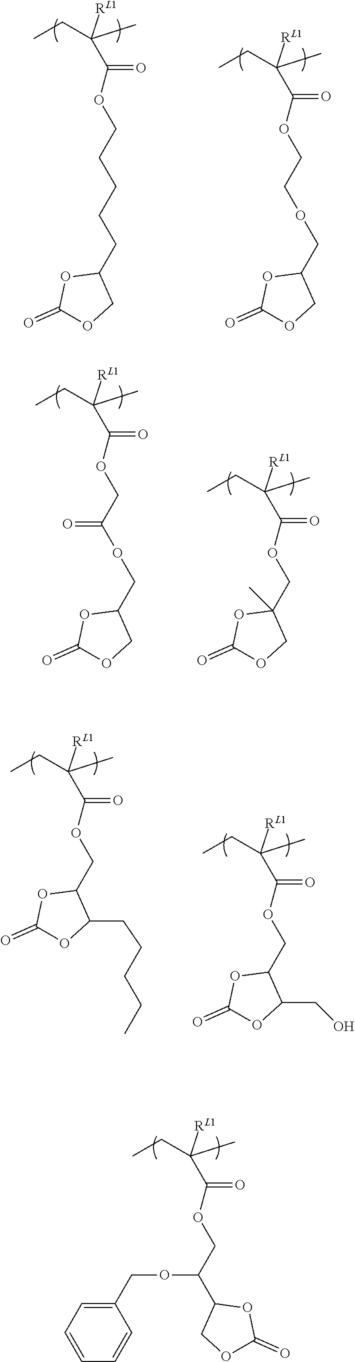

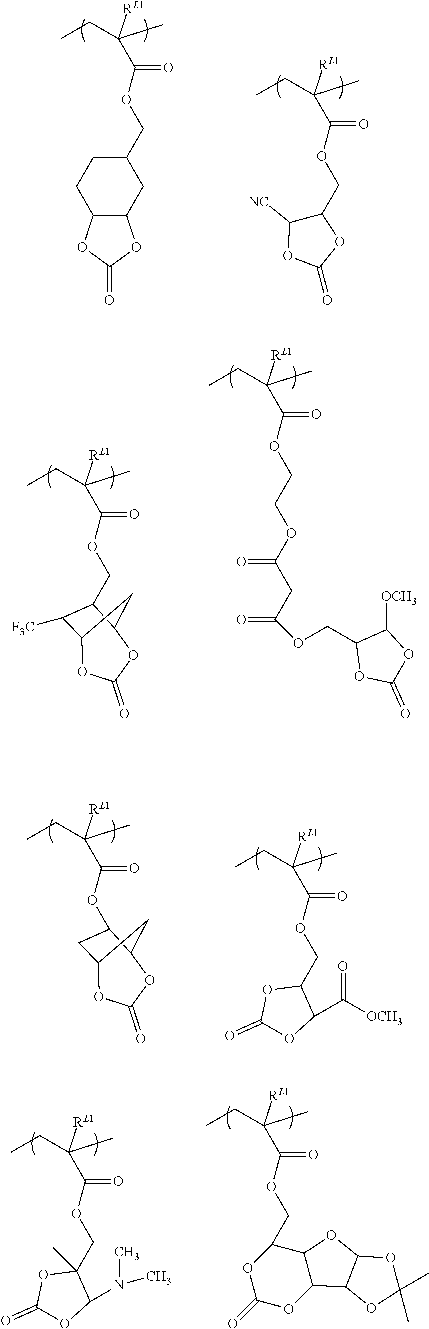

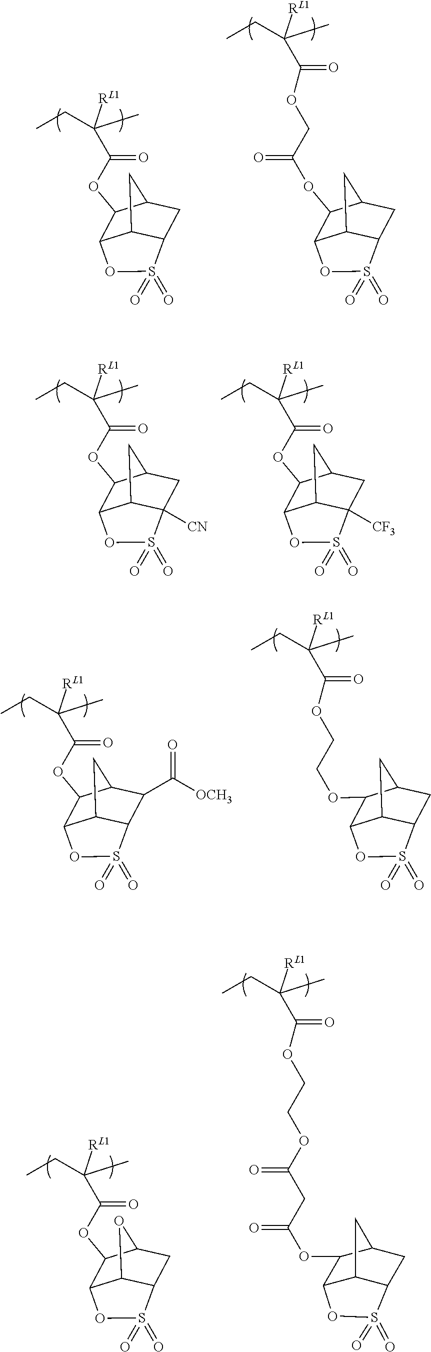

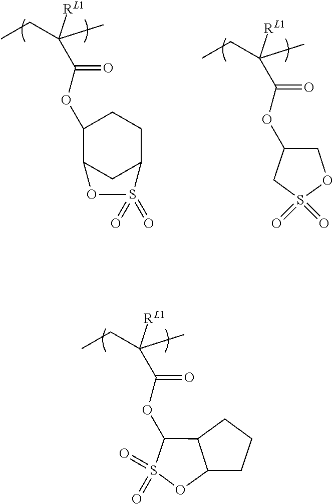

In the formulae, R represents a hydrogen atom, a fluorine atom, a methyl group, or a trifluoromethyl group.

When the polymer (P) has the structural unit (IV), a content proportion of the structural unit (IV) is preferably 1 mol % or more, and more preferably 2 mol % or more relative to the total amount of the structural units of polymer (P). The content proportion of the structural unit (IV) in the polymer (P) is preferably 30 mol % or less, more preferably 20 mol % or less, and further preferably 15 mol % or less relative to the total amount of the structural units of polymer (P).

### Structural Unit (V)

The structural unit (V) may further include a structural unit having an alcoholic hydroxy group (excluding the structural unit (I) to the structural unit (IV)). The structural unit (V) introduced into the polymer (P) can further improve the effect of inhibiting development defects when the resist pattern is formed with the present composition. The term “alcoholic hydroxy group” herein refers to a group having a structure in which a hydroxy group is directly bonded to an aliphatic hydrocarbon group. This aliphatic hydrocarbon group may be a chain hydrocarbon group or an alicyclic hydrocarbon group.

The structural unit (V) is preferably a structural unit derived from an unsaturated monomer having an alcoholic hydroxy group. The structure of the unsaturated monomer to yield the structural unit (V) is not particularly limited as long as the structure has the alcoholic hydroxy group. Specific examples of the structural unit (V) include structural units represented by the following formula.

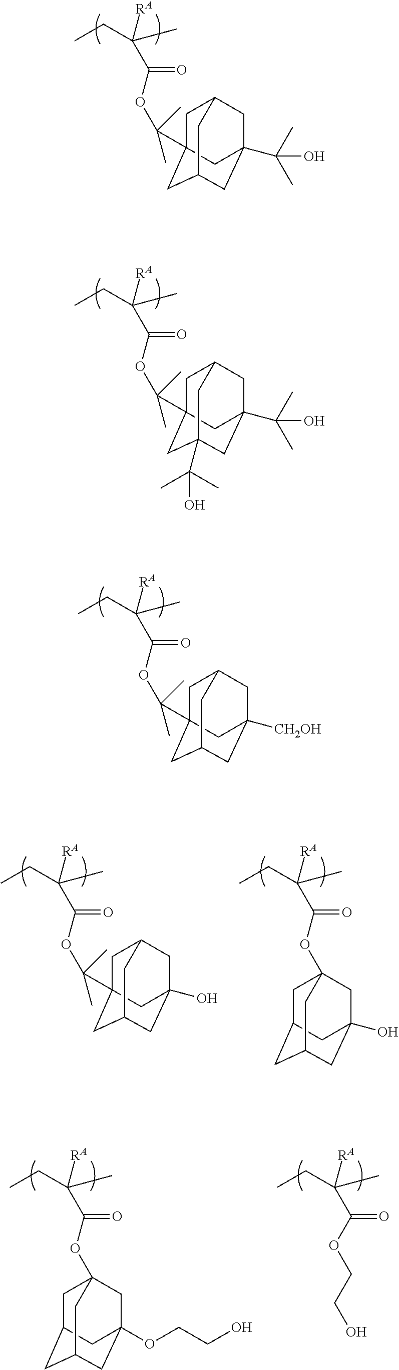

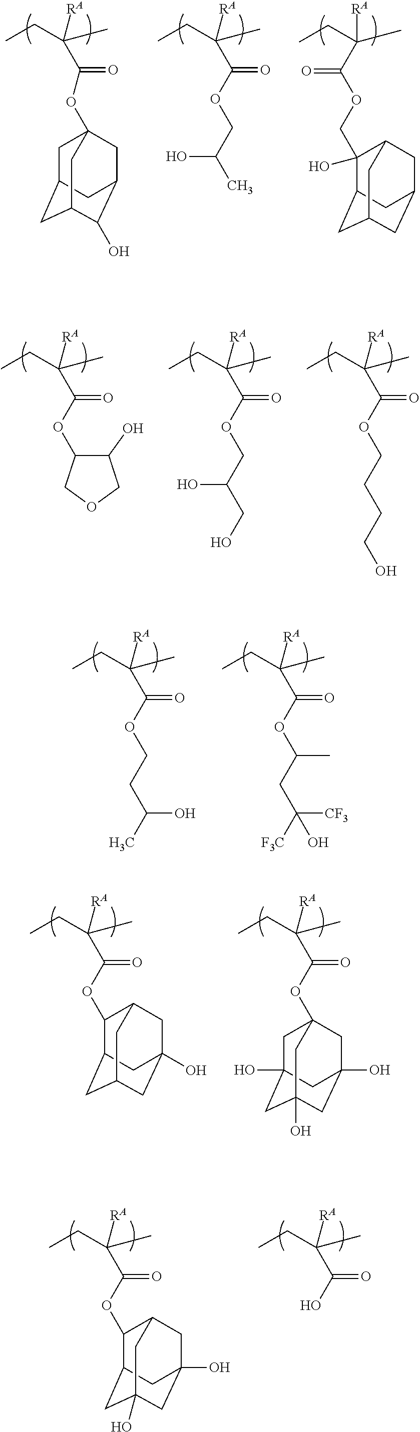

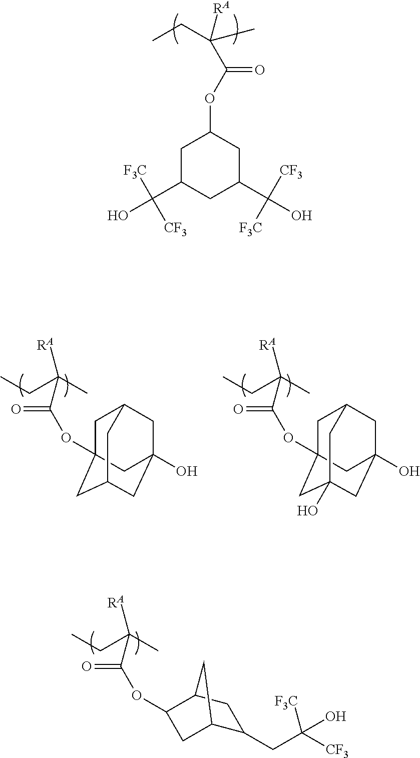

In the formula, RA represents a hydrogen atom, a fluorine atom, a methyl group, or a trifluoromethyl group.

When the polymer (P) has the structural unit (V), a content proportion of the structural unit (V) is preferably 1 mol % or more relative to the total amount of the structural units constituting the polymer (P) from the viewpoint of sufficiently yielding the effect of inhibiting development defects by introducing the structural unit (V). From the viewpoint of increasing development contrast between the exposed portion and the unexposed portion, the content proportion of the structural unit (V) is preferably 20 mol % or less, and more preferably 10 mol % or less relative to the total amount of the structural units constituting the polymer (P).

Other examples of structural units in the polymer (P) include: a structural unit having a cyano group, a nitro group, or a sulfonamide group (specifically, a structural unit derived from 2-cyanomethyladamantan-2-yl (meth)acrylate etc.); and a structural unit having a non-acid-dissociable hydrocarbon group (specifically, a structural unit derived from a substituted or unsubstituted styrene (for example, a styrene unit, a bromostyrene unit, etc.), a structural unit derived from vinylnaphthalene, and a structural unit derived from n-pentyl (meth)acrylate). A content proportion of these structural units may be appropriately set according to the structural units within a range not impairing the effect of the present invention.

In terms of ability to yield excellent sensitivity and CDU performance of the present composition, the polymer (P) preferably has an iodine atom, and more preferably has a structural unit having an iodine atom. The structural unit having an iodine atom may be the structural unit (I), or may be other structural units (for example, the structural unit (II) or the structural unit (III)).

In the polymer (P), a content proportion of the structural unit having an iodine atom is preferably 1 mol % or more, more preferably 2 mol % or more, and further preferably 5 mol % or more relative to the total amount of the structural units constituting the polymer (P) from the viewpoint of sufficiently yielding the effect of increasing sensitivity of the present composition. The content proportion of the structural unit having an iodine atom is preferably 80 mol % or less, and more preferably 70 mol % or less relative to the total amount of the structural units constituting the polymer (P). In the structural unit having an iodine atom, the iodine atom is preferably bonded to an aromatic ring.

A weight-average molecular weight (Mw) of the polymer (P) in terms of polystyrene by gel permeation chromatography (GPC) is preferably 1,000 or more, more preferably 2,000 or more, further preferably 3,000 or more, and furthermore preferably 4,000 or more. Mw of the polymer (P) is preferably 50,000 or less, more preferably 30,000 or less, further preferably 20,000 or less, and furthermore preferably 15,000 or less. Setting Mw of the polymer (P) to be within the above range is preferable in terms of ability to improve coatability of the present composition and sufficiently inhibit development defects.

A ratio (Mw/Mn, hereinafter, also referred to as “dispersion degree”) of the polymer (P) of Mw to a number-average molecular weight (Mn) in terms of polystyrene by GPC is preferably 5.0 or less, more preferably 3.0 or less, and further preferably 2.0 or less. Mw/Mn is typically 1.0 or more.

The polymer (P) preferably constitutes a base resin of the present composition. The term “base resin” herein refers to a component accounting for more than 50 mass % relative to the total amount of solid contents contained in the present composition. The term “the total amount of solid contents” herein refers to a total of components other than a solvent. A content of the polymer (P) in the present composition is preferably 60 mass % or more, more preferably 70 mass % or more, and further preferably 85 mass % or more relative to the total amount of the solid contents contained in the present composition.

### [Radiation-Sensitive Acid Generating Substance]

### Acid Generator

For a purpose of further increasing sensitivity and CDU performance of the present composition etc., the present composition may contain an acid generator as the radiation-sensitive acid generating substance. The acid generator generates a strong acid by exposure, which acid induces dissociation of the acid-dissociable group under a normal condition. Note that “normal condition” herein refers to a condition of performing post exposure baking (PEB) at 110° C. for 60 seconds. The acid generator is a component different from the polymer (P). A molecular weight of the acid generator is preferably 1,000 or less, more preferably 800 or less, and further preferably 600 or less.

The type of the acid generator blended in the present composition is not particularly limited, and a known radiation-sensitive acid generator used for resist pattern formation may be appropriately used. The acid generator may be an ionic radiation-sensitive acid generator or may be a nonionic radiation-sensitive acid generator. The acid generator is preferably an ionic radiation-sensitive acid generator, and is an onium salt composed of a radiation-sensitive onium cation and an organic anion, for example. The acid generator is preferably a compound that generates an acid (preferably a strong acid such as a sulfonic acid, an imide acid, and a methide acid) in the composition under the aforementioned normal condition. The acid has acidity higher than the acid generated from the acid-diffusion inhibitor (more specifically, the photodegradable base) and consequently induces dissociation of the acid-dissociable group with this acid.

When the onium salt is used as the acid generator, the acid generator preferably has a sulfonium cation or an iodonium cation, and more preferably has an arylsulfonium cation or an aryliodonium cation from the viewpoint of increasing sensitivity of the present composition and the viewpoint of forming the resist film having more excellent lithography performance. Specific examples of these cations include the cation represented by the formula (4), the cation represented by the formula (5), and the cation represented by the formula (6). Further specific examples of the radiation-sensitive onium cation in the acid generator include cations same as described as the specific examples of the cation represented by the formula (4), the cation represented by the formula (5), and the cation represented by the formula (6). Note that the sulfonium cation when the formula (4) or the formula (6) has an iodo group as a substituent and the iodonium cation represented by the formula (5) are also specific examples of the onium cation in the iodine-containing onium salt when the acid generator is the iodine-containing onium salt. Among these, the sulfonium cation having an aromatic ring substituted with an iodine atom and the iodonium cation having an aromatic ring to which an iodo group is bonded are preferable in terms of ability to yield the radiation-sensitive composition having higher sensitivity while inhibiting development defects.

The organic anion in the acid generator is typically an anion in which a proton is removed from an acid group in an organic acid. The organic anion is not particularly limited, and preferably a sulfonate anion, an imide anion, or a methide anion in terms of ability to increase sensitivity of the present composition. Specific examples of the sulfonate anion include anions represented by the following formulae. Note that the anions having an iodine atom among the following formulae are also specific examples of the organic anion in the iodine-containing onium salt when the acid generator is the iodine-containing onium salt.

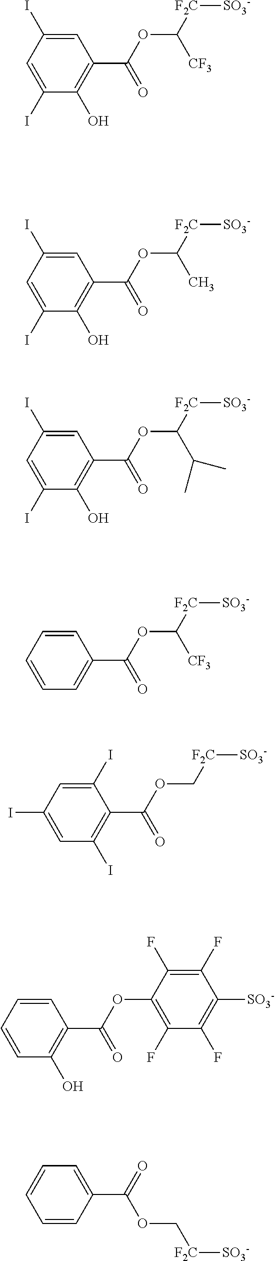

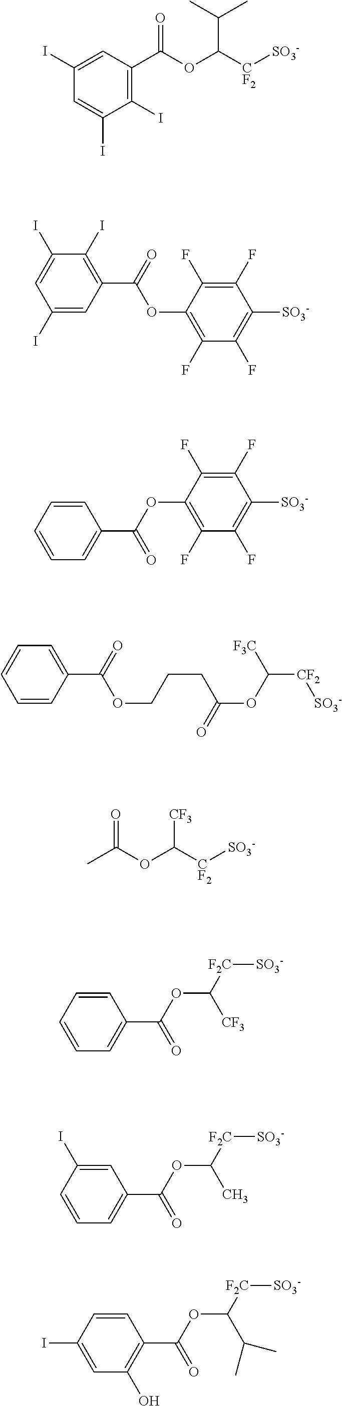

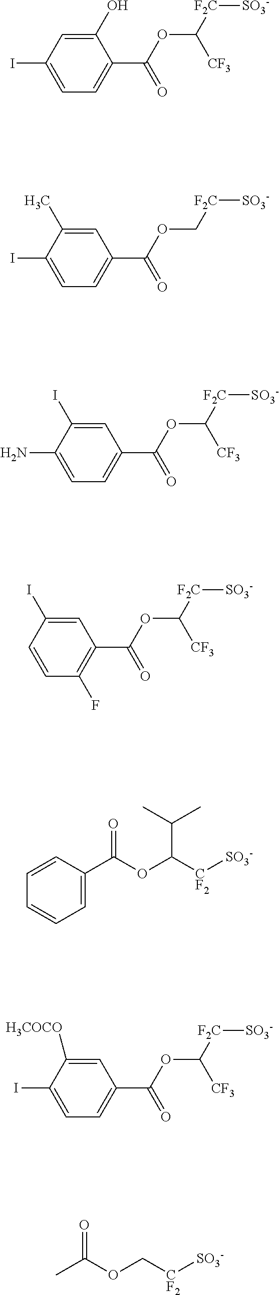

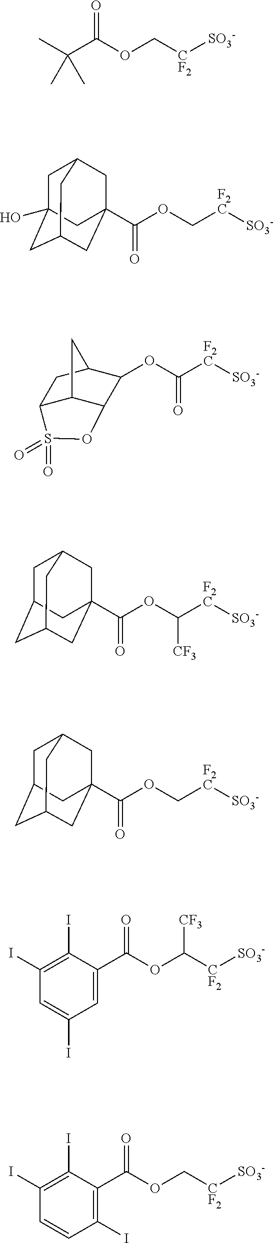

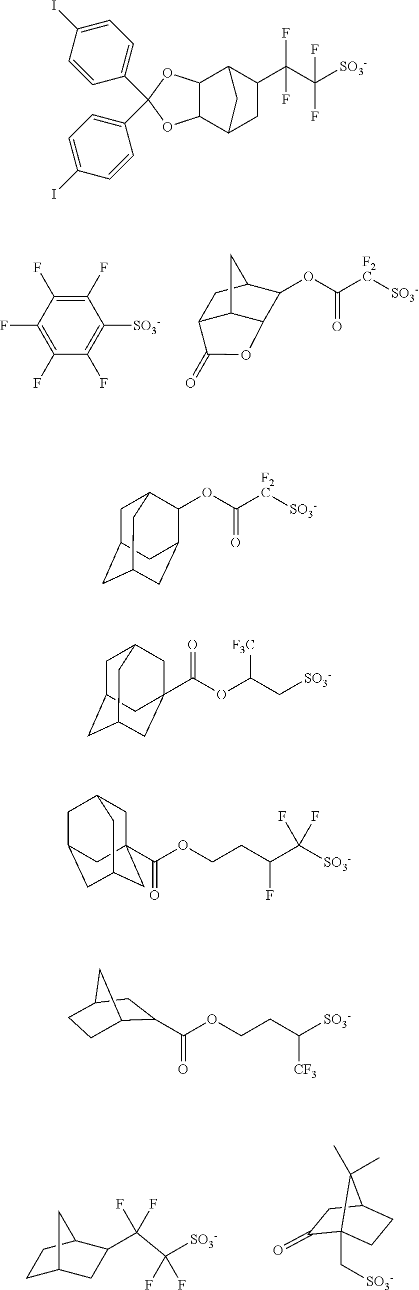

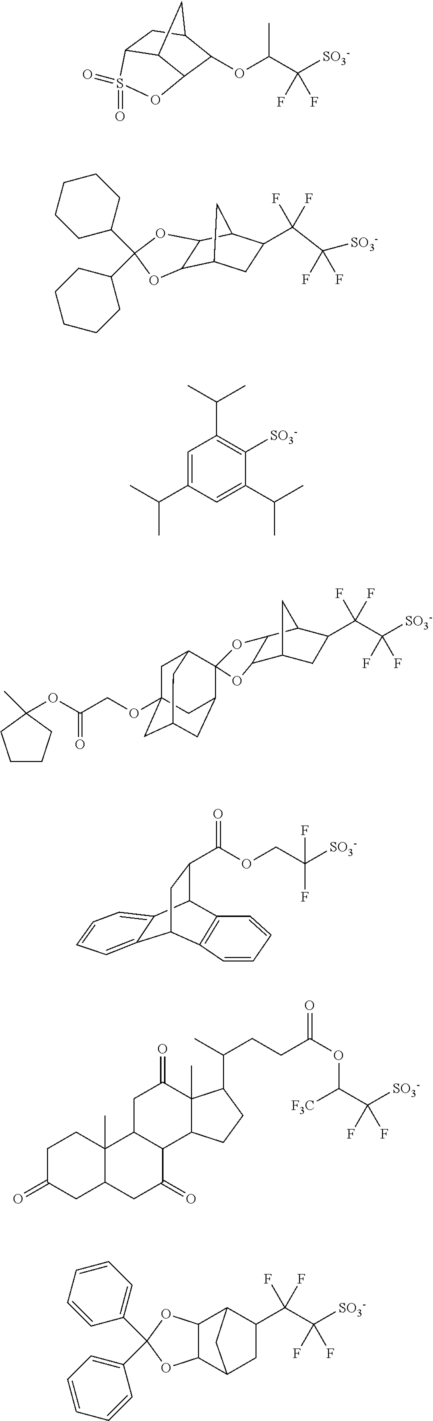

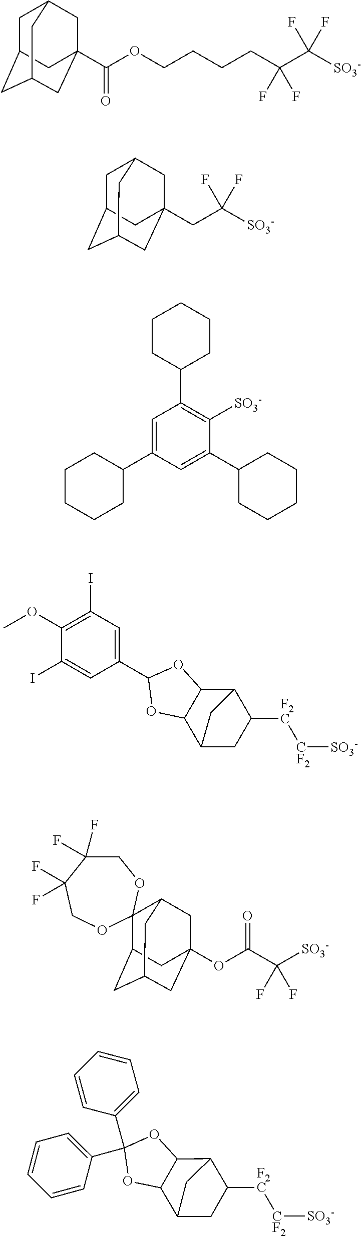

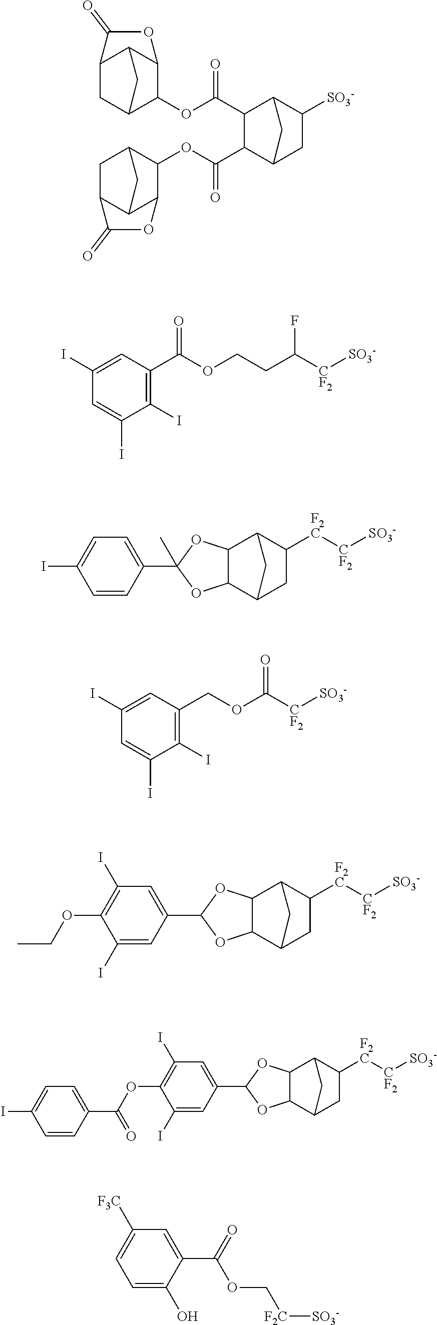

When the acid generator is blended in the present composition, a content proportion of the acid generator is preferably 0.5 parts by mass or more, more preferably 1 part by mass or more, and further preferably 2 parts by mass or more relative to 100 parts by mass of the polymer (P) from the viewpoint of sufficiently yielding the effect of improving sensitivity by blending the acid generator. From the viewpoint of inhibiting occurrence of development defects caused by the acid generator, the content proportion of the acid generator is preferably 30 parts by mass or less, more preferably 25 parts by mass or less, and further preferably 20 parts by mass relative to 100 parts by mass of the polymer (P).

When the acid generator is blended in the present composition, an onium salt having an iodine atom (namely, the iodine-containing onium salt) can be preferably used as the acid generator in terms of ability to improve sensitivity, CDU performance, and development defect inhibition of the present composition with good balance. In the acid generator having an iodine atom, the number of the iodine atoms in one molecule is preferably two or more, and more preferably three or more from the viewpoint of increasing CDU performance of the radiation-sensitive composition.

### Acid-Diffusion Inhibitor

The acid-diffusion inhibitor is a substance that can inhibit chemical reactions by an acid in the unexposed portion by inhibiting diffusion, in the resist film, of the acid generated by exposing the present composition in the resist film. The acid-diffusion inhibitor generates a weak acid by exposure, which acid does not induce dissociation of the acid-dissociable group under the aforementioned normal condition. Such an acid-diffusion inhibitor may be blended in the present composition to further improve lithography performance of the present composition. The acid-diffusion inhibitor herein is a component different from the polymer (P). A molecular weight of the acid-diffusion inhibitor is preferably 1,000 or less, more preferably 800 or less, and further preferably 600 or less.

As the acid-diffusion inhibitor, an onium salt composed of an onium cation (more specifically, a radiation-sensitive onium cation) and an organic anion (hereinafter, also referred to as “photodegradable base”) can be preferably used. From the viewpoint of improving lithography performance of the present composition, the present composition preferably contains the photodegradable base as the radiation-sensitive acid generating substance. The photodegradable base is preferably an onium salt to generate a carboxylic acid, a sulfonic acid, or a sulfonamide by exposure. In terms of ability to forming the resist film having higher lithography performance, an onium salt having a sulfonium cation or an iodonium cation can be preferably used as the photodegradable base.

Specific examples of the onium cation in the photodegradable base include the cation represented by the formula (4), the cation represented by the formula (5), and the cation represented by the formula (6). Further specific examples of the onium cation in the photodegradable base include cations same as described as the specific examples of the cation represented by the formula (4), the cation represented by the formula (5), and the cation represented by the formula (6). The sulfonium cation represented by the formula (4) or the formula (6) having an iodo group as a substituent and the iodonium cation represented by the formula (5) are also specific examples of the onium cation in the iodine-containing onium salt when the photodegradable base is the iodine-containing onium salt. Among these, the onium cation in the photodegradable base is preferably a sulfonium cation having an aromatic ring substituted with an iodine atom or an iodonium cation having an aromatic ring to which an iodo group is bonded.

Examples of the organic anion in the photodegradable base include anions represented by the following formulae. In the following formulae, the anions having an iodine atom are also specific examples of the organic anion in the iodine-containing onium salt when the photodegradable base is the iodine-containing onium salt. The organic anion in the photodegradable base is preferably a carboxylate anion having an aromatic ring substituted with an iodine atom.

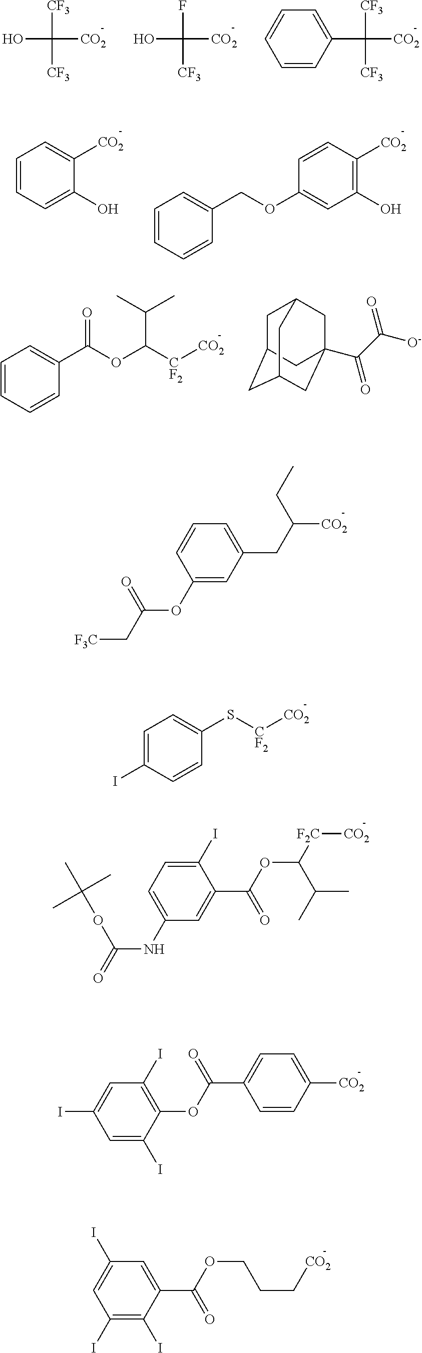

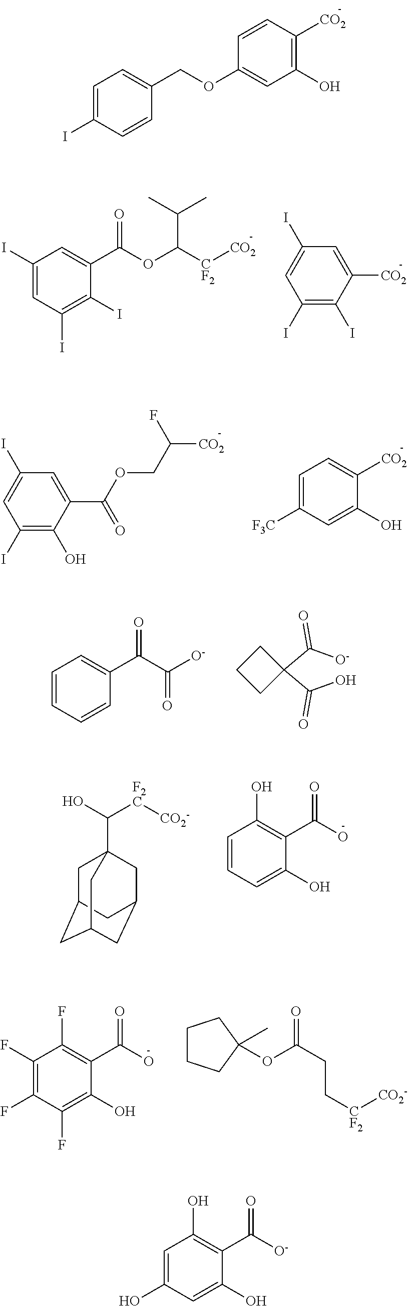

When the acid-diffusion inhibitor is blended in the present composition, a content proportion of the acid-diffusion inhibitor is preferably 1 part by mass or more, and more preferably 2 parts by mass or more relative to 100 parts by mass of the polymer (P) from the viewpoint of sufficiently yielding the effect of improving sensitivity and lithography performance by blending the acid-diffusion inhibitor. From the viewpoint of inhibiting occurrence of development defects caused by the acid-diffusion inhibitor, the content proportion of the acid-diffusion inhibitor is preferably 30 parts by mass of less, and more preferably 20 parts by mass or less relative to 100 parts by mass of the polymer (P).

When the acid-diffusion inhibitor is blended in the present composition, the content proportion of the acid-diffusion inhibitor in the present composition is preferably 2 mol % or more, more preferably 5 mol % or more, and further preferably 10 mol % or more relative to a total amount of the acid generator and the monomer to yield the structural unit (I) contained in the present composition. The content proportion of the acid-diffusion inhibitor is preferably 90 mol % or less, more preferably 80 mol % or less, and further preferably 70 mol % or less relative to a total amount of the acid generator and the monomer to yield the structural unit (I) contained in the present composition. Setting the content proportion of the acid-diffusion inhibitor to be within the above range can more increase CDU performance of the present composition.

Preferable specific aspects of the polymer (P) and the radiation-sensitive acid generating substance in the present composition satisfying one or more of the requirement 1, the requirement 2, and the requirement 3 include the following aspects, for example.

Aspect 1: an aspect containing the polymer (P) and the acid-diffusion inhibitor, and optionally containing the acid generator, wherein the structural unit (I) in the polymer (P) has two or more iodine atoms.

Aspect 2: an aspect containing the polymer (P) and the acid-diffusion inhibitor, and optionally containing the acid generator, wherein the acid-diffusion inhibitor has two or more iodine atoms.

Aspect 3: an aspect containing the polymer (P), the acid generator, and the acid-diffusion inhibitor, wherein the acid generator has two or more iodine atoms.

Aspect 4: an aspect containing the polymer (P) and the acid-diffusion inhibitor, and optionally containing the acid generator, wherein the structural unit (I) in the polymer (P) has one or more iodine atoms, and the acid-diffusion inhibitor has one or more iodine atoms.

Aspect 5: an aspect containing the polymer (P), the acid generator, and the acid-diffusion inhibitor, wherein the structural unit (I) in the polymer (P) has one or more iodine atoms, and the acid generator has one or more iodine atoms.

The above aspects 1 to 5 may further contain another component. In the aspect 1, the acid-diffusion inhibitor may have an iodine atom. In the aspect 2, the structural unit (I) in the polymer (P) may have an iodine atom. In the aspects 1, 2, and 4, the optionally contained acid generator may have an iodine atom.

### [Other Components]

The present composition may further contain a component different from the polymer (P) and the radiation-sensitive acid generating substance (hereinafter, also referred to as “other component”) in addition to the polymer (P) and the radiation-sensitive acid generating substance. Examples of the other component include a solvent and a high-fluorine-content polymer.

### (Solvent)

The solvent is preferably a solvent that can dissolve or disperse the components blended in the present composition, and an organic solvent may be preferably used. Specific examples of the solvent include alcohols, ethers, ketones, amides, esters, and hydrocarbons.

Examples of the alcohols include: aliphatic monohydric alcohols having 1 to 18 carbon atoms such as 4-methyl-2-pentanol and n-hexanol; alicyclic monohydric alcohols having 3 to 18 carbon atoms such as cyclohexanol; polyhydric alcohols having 2 to 18 carbon atoms such as 1,2-propylene glycol; and polyhydric alcohol partial ethers having 3 to 19 carbon atoms such as propylene glycol monomethyl ether. Examples of the ethers include: dialkyl ethers such as diethyl ether, dipropyl ether, dibutyl ether, dipentyl ether, diisoamyl ether, dihexyl ether, and diheptyl ether; cyclic ethers such as tetrahydrofuran and tetrahydropyran; and aromatic-ring-containing ethers such as diphenyl ether and anisole.

Examples of the ketones include: chain ketones such as acetone, methyl ethyl ketone, methyl n-propyl ketone, methyl n-butyl ketone, diethyl ketone, methyl iso-butyl ketone, 2-heptanone, ethyl n-butyl ketone, methyl n-hexyl ketone, di-iso-butyl ketone, and trimethylnonanone; cyclic ketones such as cyclopentanone, cyclohexanone, cycloheptanone, cyclooctanone, and methylcyclohexanone; and 2,4-pentanedione, acetonylacetone, acetophenone, and diacetone alcohol. Examples of the amides include: cyclic amides such as N,N′-dimethylimidazolidinone and N-methylpyrrolidone; and chain amides such as N-methylformamide, N,N-dimethylformamide, N,N-diethylformamide, acetamide, N-methylacetamide, N,N-dimethylacetamide, and N-methylpropionamide.

Examples of the esters include: monocarboxylic acid esters such as n-butyl acetate, ethyl lactate, and methyl 2-hydroxyisobutyrate; polyhydric alcohol carboxylates such as propylene glycol diacetate; polyhydric alcohol partial ether carboxylates such as propylene glycol monomethyl ether acetate; polyvalent carboxylic acid diesters such as diethyl oxalate; carbonates such as dimethyl carbonate and diethyl carbonate; and cyclic esters such as γ-butyrolactone. Examples of the hydrocarbons: include aliphatic hydrocarbons having 5 to 12 carbon atoms such as n-pentane and n-hexane; and aromatic hydrocarbons having 6 to 16 carbon atoms such as toluene and xylene.

Among these, the solvent preferably contains at least one selected from the group consisting of the esters and ketones, and more preferably contains at least one selected from the group consisting of the polyhydric alcohol partial ether carboxylates and the cyclic ketones.

### (High-Fluorine-Content Polymer)

The high-fluorine-content polymer (hereinafter, also referred to as “polymer (F)”) is a polymer having a higher mass content rate of a fluorine atom than the polymer (P). The polymer (F) is contained in the present composition as a water-repellent additive, for example.

The fluorine atom content rate of the polymer (F) is not particularly limited as long as the content is higher than that of the polymer (P). The fluorine atom content rate of the polymer (F) is preferably 1 mass % or more, and more preferably 4 mass % or more, and further preferably 7 mass % or more. The fluorine atom content rate of the polymer (F) is preferably 60 mass % or less, and more preferably 40 mass % or less. It is noted that the fluorine atom content rate (mass %) of the polymer can be calculated from a structure of the polymer determined by ¹³C-NMR spectrum measurement etc.

When the present composition contains the polymer (F), a content of the polymer (F) in the present composition is preferably 0.05 part by mass or more, more preferably 0.1 parts by mass of more, and further preferably 0.5 part by mass or more relative to 100 parts by mass of the polymer (P). The content of the polymer (F) is preferably 10 parts by mass or less, and more preferably 5 parts by mass or less relative to 100 parts by mass of the polymer (P).

### (Other Optional Components)

The present composition may further contain a component different from the above polymer (P), the radiation-sensitive acid generating substance, the solvent, and the polymer (F) (hereinafter, also referred to as “other optional component”). Examples of the other optional component include a surfactant, an alicyclic-skeleton-containing compound (for example, 1-adamantanecarboxylic acid, 2-adamantanone, t-butyl deoxycholate, etc.), a sensitizer, and an uneven-distribution enhancer. A content of the other optional components may be appropriately set according to each compound within the range not impairing the effect of the present invention.

### <Method for Manufacturing Radiation-Sensitive Composition>

The present composition can be manufactured by, for example, mixing the polymer (P), the radiation-sensitive acid generating substance, and components such as the solvent as necessary at a desired ratio, and filtering the obtained mixture preferably by using a filter (for example, a filter with a pore diameter of about 0.2 μm). A solid-content concentration of the present composition is preferably 0.1 mass % or more, more preferably 0.5 mass % or more, and further preferably 1 mass % or more. The solid-content concentration of the present composition is preferably 50 mass % or less, more preferably 20 mass % or less, and further preferably 5 mass % or less. Setting the solid-content concentration of the present composition to be within the above range is preferable in terms of ability to provide good coatability to improve a shape of the resist pattern.

The present composition obtained as above can be used as a composition for positive-type pattern formation, which forms a pattern using an alkali developer liquid, or can be used as a composition for negative-type pattern formation using a developer liquid containing an organic solvent.

### <<Polymer>>

The present disclosure provides a polymer (hereinafter, also referred to as “polymer P1”) including a partial structure, wherein Y¹ in the formula (1) represents a divalent group represented by the following formula (2-1):

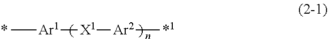

wherein Ar¹ represents a divalent aromatic ring group, Ar² represents a single bond or a divalent aromatic ring group, X¹ represent a divalent linking group, “n” represents 0 or 1, “*¹” represents an attachment point to a carbonyl group in the formula (1), and “*” represents an attachment point;
R¹ represents a divalent hydrocarbon group; and M⁺ represents a sulfonium cation or an iodonium cation,
wherein the partial structure represented by the formula (1) has two or more iodine atoms.

The present disclosure provides a polymer (hereinafter, also referred to as “polymer P2”) including a partial structure, wherein Y¹ in the formula (1) represents a divalent group represented by the following formula (2-2):

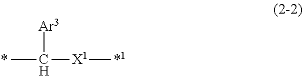

wherein Ar³ represents a monovalent aromatic ring group,
X² represents a divalent linking group, “*¹” represents an attachment point to a carbonyl group in the formula (1), and “*” represents an attachment point,
R¹ represents a divalent hydrocarbon group; and M⁺ represents a sulfonium cation or an iodonium cation.

These polymer P1 and polymer P2 are suitable as a polymer component (specifically, a base resin) of the radiation-sensitive composition used for the resist pattern formation.

### <<Compound>>

The present disclosure provides a compound represented by the following formula (3-1) (hereinafter, also referred to as “compound (3-1)”)

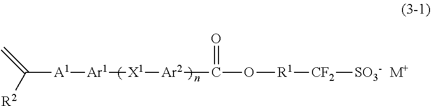

In the formula (3-1), R² represents a hydrogen atom, a fluorine atom, a methyl group, or a trifluoromethyl group. A¹ represents a single bond or *³—CO—O—. “*³” represents an attachment point to the carbon atom to which R² is bonded. Ar¹ represents a divalent aromatic ring group. Ar² represents a single bond or a divalent aromatic ring group. X¹ represents a divalent linking group. “n” represents 0 or 1. R¹ represents a divalent hydrocarbon group. M⁺ represents a sulfonium cation or an iodonium cation. The compound has two or more iodine atoms in the formula (3-1).

The present disclosure provides a compound represented by the following formula (3-2) (hereinafter, also referred to as “compound (3-2)”).

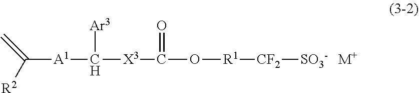

In the formula (3-2), R² represents a hydrogen atom, a fluorine atom, a methyl group, or a trifluoromethyl group. A¹ represents a single bond or *³—CO—O—. “*³” represents an attachment point to the carbon atom to which R² is bonded. Ar³ represents a monovalent aromatic ring group. X² represents a divalent linking group. R¹ represents a divalent hydrocarbon group. M⁺ represents a sulfonium cation or an iodonium cation.

These compound (3-1) and compound (3-2) are suitable as a monomer to constitute the polymer component (specifically, base resin) of the radiation-sensitive composition used for the resist pattern formation. A method for synthesizing the compound (3-1) and the compound (3-2) is not particularly limited, and the compound (3-1) and the compound (3-2) can be manufactured by appropriately combining common methods of organic chemistry. An example of the method for synthesizing the compound (3-1) is as follows: a compound having a partial structure corresponding to “CH₂=CR²-A¹-Ar¹—(X¹—Ar²)ₙ)—COO—” in the formula (3-1) and a compound composed of an anion having a partial structure corresponding to —R¹—CF₂—SO₃— in the formula (3-1) and a cation (for example, an ammonium cation) are reacted in an appropriate solvent in the presence of a catalyst as necessary to obtain an intermediate product; and then, the obtained intermediate product and a sulfonium halide, an iodonium halide, etc. to provide the onium cation moiety are reacted to obtain the compound represented by the formula (3-1). Note that the method for synthesizing the compound (3-1) and the compound (3-2) is not limited to the above.

### <<Method for Forming Resist Pattern>>

A method for forming a resist pattern of the present disclosure includes: a step of applying the present composition on one surface of a substrate (hereinafter, also referred to as “applying step”); a step of exposing the resist film obtained in the applying step (hereinafter, also referred to as “exposing step”); and a step of developing the resist film exposed in exposing step (hereinafter, also referred to as “developing step”). Examples of the pattern formed by the method for forming a resist pattern of the present disclosure include a line-and-space pattern and a hole pattern. The method for forming a resist pattern of the present disclosure uses the present composition to form the resist film, and thereby the resist pattern having good sensitivity and CDU performance and reduced development defects can be formed. Hereinafter, each step will be described.

### [Applying Step]

In the applying step, the present composition is applied on one surface of a substrate to form a resist film on the substrate. As the substrate on which the resist film is to be formed, conventional substrate may be used. Examples thereof include a silicon wafer, silicon dioxide, and a wafer coated with aluminum. For example, an organic or inorganic anti-reflective film, described in Japanese Patent Laid-Open No. S59-93448, etc., may be formed on the substrate. Examples of a method for applying the present composition include spin coating, casting coating, and roll coating. After application, soft baking (hereinafter, also referred to as “SB”) to evaporate the solvent in the coating film may be performed. The temperature of SB is preferably 60° C. or higher, and more preferably 80° C. or higher. The temperature of SB is preferably 140° C. or lower, and more preferably 120° C. or lower. The time of SB is preferably 5 seconds or longer, and more preferably 10 seconds or longer. The time of SB is preferably 600 seconds or shorter, and more preferably 300 seconds or shorter. An average thickness of the resist film to be formed is preferably 10 to 1,000 nm, and more preferably 20 to 500 nm. Note that the soft baking is also referred to as “pre-baking”.

### [Exposing Step]

In the exposing step, the resist film obtained in the applying step is exposed. This exposure is performed by irradiating the resist film with radiation through a photomask, or through a liquid-immersion medium such as water in some cases. Examples of the radiation include: electromagnetic wave such as visible light ray, ultraviolet ray, far ultraviolet ray, extreme ultraviolet ray (EUV), X-ray, and γ-ray; and charged particle beam such as electron beam and α-ray, according to a line width of the target pattern. Among these, the radiation for irradiating the resist film formed by using the present composition is preferably far ultraviolet rays, EUV, or electron beams, more preferably ArF excimer laser light (wavelength: 193 nm), KrF excimer laser light (wavelength: 248 nm), EUV, or electron beams, further preferably ArF excimer laser light, EUV, or electron beams, furthermore preferably EUV or electron beams, and particularly preferably EUV.

After the exposure, post exposure baking (PEB) is preferably performed to enhance dissociation of the acid-dissociable group by the acid generated from the compound that generates the acid (such as the radiation-sensitive acid generator) by exposure in the exposed portion of the resist film. This PEB can increase a difference in solubility in the developer liquid between the exposed portion and the unexposed portion. The temperature of PEB is preferably 50° C. or higher, and more preferably 80° C. or higher. The temperature of PEB is preferably 180° C. or lower, and more preferably 130° C. or lower. The time of PEB is preferably 5 seconds or longer, and more preferably 10 seconds or longer. The time of PEB is preferably 600 seconds or shorter, and more preferably 300 seconds or shorter.

### [Developing Step]

In the developing step, the exposed resist film is developed. This development can form the desired resist pattern. After the development, washing with a rinse liquid such as water and an alcohol, and drying are typically performed. A development method in the developing step may be alkali development or may be organic-solvent development.

In the case of the alkali development, examples of the developer liquid for the development include an alkali aqueous solution dissolving at least one of alkaline compounds such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (TMAH), pyrrole, piperidine, choline, 1,8-diazabicyclo-[5.4.0]-7-undecene, and 1,5-diazabicyclo-[4.3.0]-5-nonene. Among these, a TMAH aqueous solution is preferable, and a 2.38 mass % TMAH aqueous solution is more preferable. In the case of the organic-solvent development, examples of the developer liquid include one or more organic solvents such as hydrocarbons, ethers, esters, ketones, and alcohols, and solvents containing the organic solvent.

Examples of the development method include a method of immersing the substrate in a vessel filled with the developer liquid for a certain time (dip method), a method of lifting up the developer liquid to the substrate surface with a surface tension and leaving to stand the developer liquid for a certain time (puddle method), a method of spraying the developer liquid onto the substrate surface (spraying method), and a method of continuously discharging the developer liquid onto the substrate rotating at a certain speed while scanning the developer-liquid discharging nozzle at a certain speed (dynamic dispense method).

### EXAMPLES

Hereinafter, the present invention will be specifically described with showing Synthesis Examples, Examples, and Comparative Examples, but the present invention is not limited to the following Examples. Note that “parts” and “%” in the following examples are mass basis unless otherwise mentioned.

### <Synthesis of Radiation-Sensitive Acid Generator>

### Synthesis Example A1: Synthesis of Compound (A-1)

A compound (A-1) was synthesized according to the following reaction scheme.

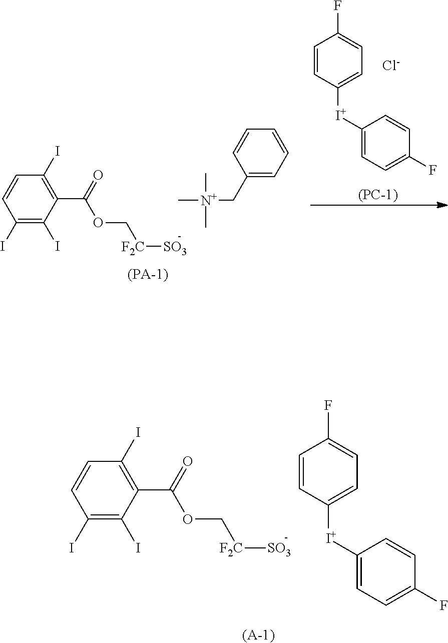

Into a round-bottom flask, a compound (PA-1) (20 mmol), a compound (PC-1) (20 mmol), methylene chloride (50 mL), and water (50 mL) were added, and the mixture was stirred at room temperature for 3 hours. The organic layer was washed three times with water, and then the organic layer was dried with sodium sulfate and filtered. The solvent was distilled off to obtain the compound (A-1).

### Synthesis Examples A2 to A12: Synthesis of Compounds (A-2) to (A-12)

The precursor was appropriately selected and a procedure similar to that in Synthesis Example A1 was selected to synthesize radiation-sensitive acid generators represented by the following formulae (A-1) to (A-12) (compounds (A-2) to (A-12)).

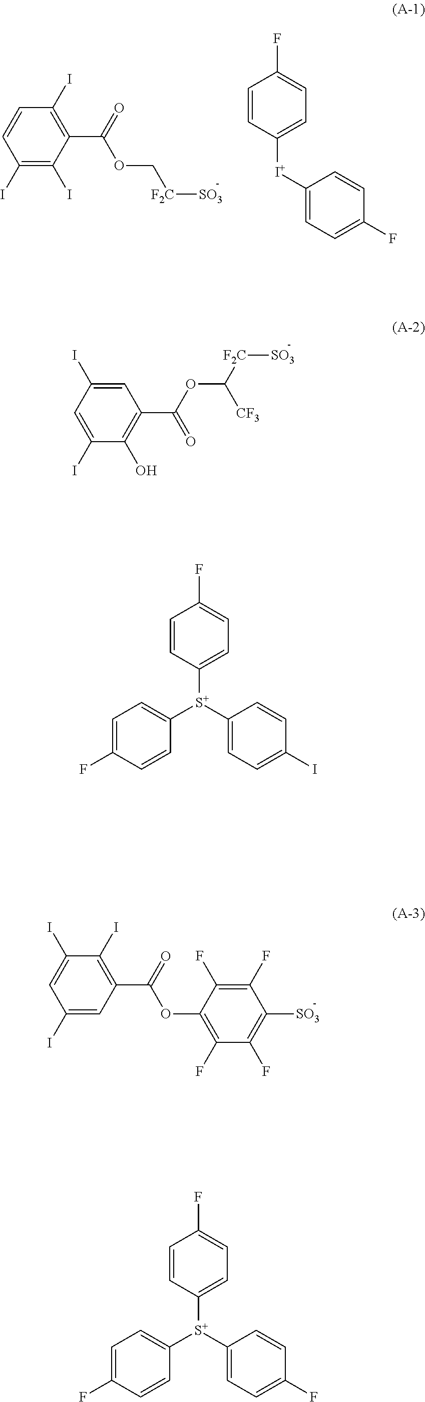

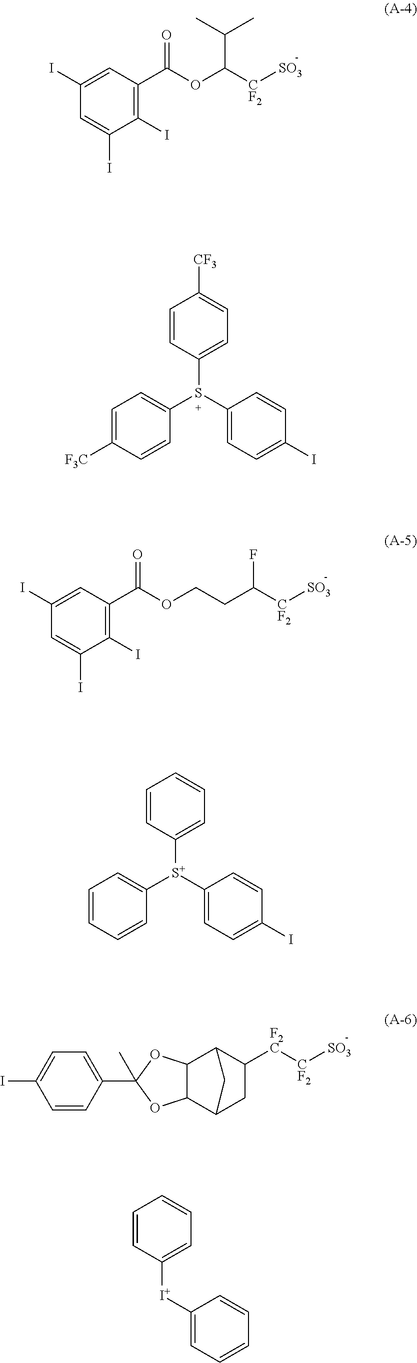

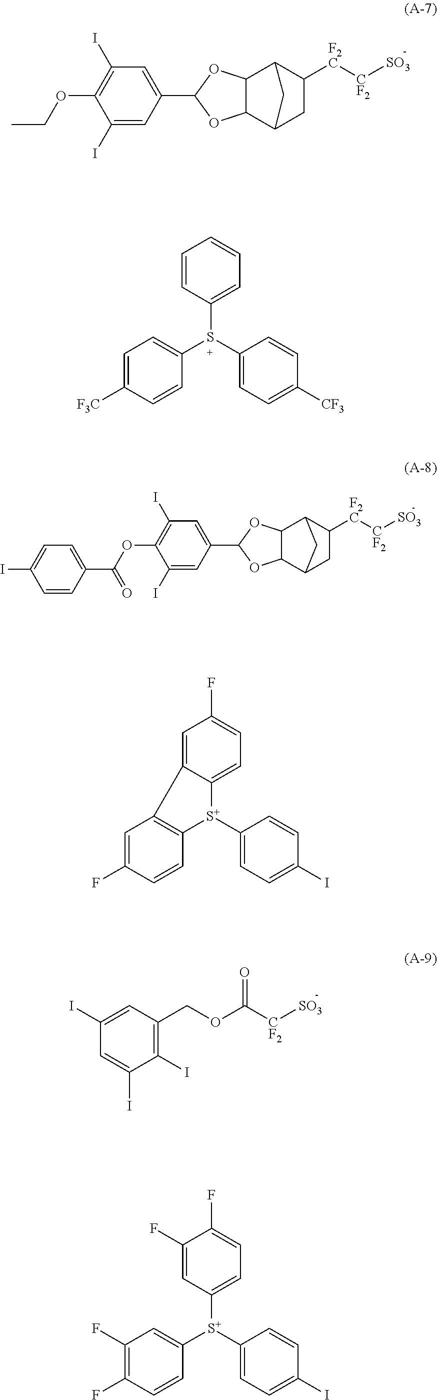

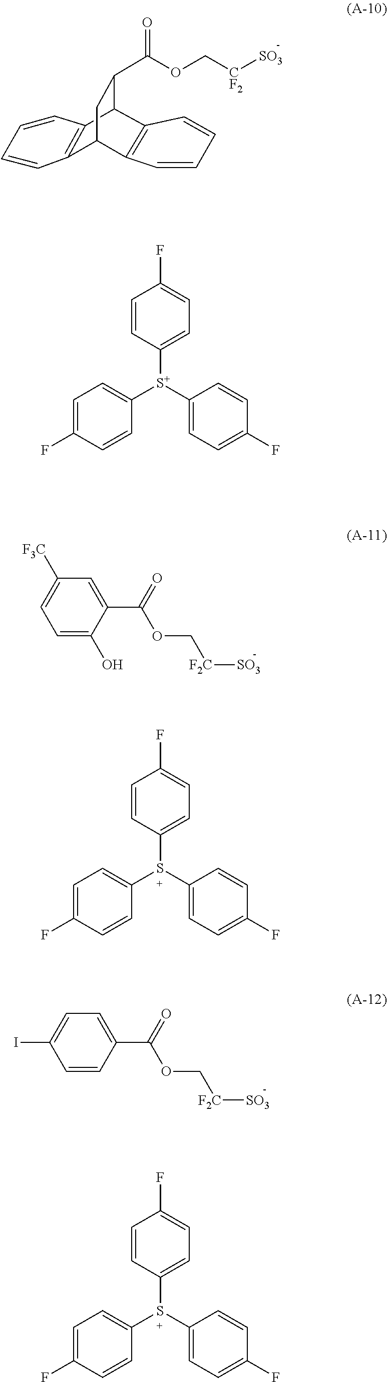

### <Synthesis of Radiation-Sensitive Acid Generator Monomer>

### Synthesis Example B1: Synthesis of Compound (B-1)

A compound (B-1) was synthesized according to the following reaction scheme.

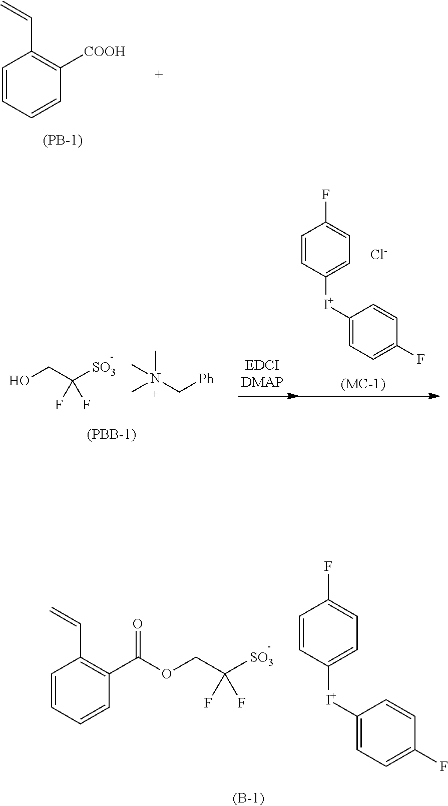

Into a round-bottom flask, a compound (PB-1) (84 mmol) and acetonitrile (70 mL) were added, 1-(3-dimethylaminopropyl)-3-ethylcarbodiimide (84 mmol) was further added, and the mixture was cooled with ice. A compound (PBB-1) (100 mmol) and 4-dimethylaminopyridine (17 mmol) were added, and the mixture was stirred at room temperature. Methylene chloride (180 mL) was added, and then the mixture was washed with a 2-mol/L hydrochloric acid aqueous solution and ultrapure water. Ultrapure water (200 mL) and (MC-1) (84 mmol) were added, and the mixture was stirred at room temperature for 2 hours. The organic layer was washed three times with water, and the organic layer was dried with sodium sulfate and filtered. The solvent was distilled off, and the residue was purified by silica gel chromatography to obtain the compound (B-1).

### Synthesis Examples B2 to B13: Synthesis of Compounds (B-2) to (B-13)

The precursor was appropriately selected and a procedure similar to that in Synthesis Example B1 was selected to synthesize radiation-sensitive acid generator monomers represented by the following formulae (B-1) to (B-13) (compounds (B-2) to (B-13)).

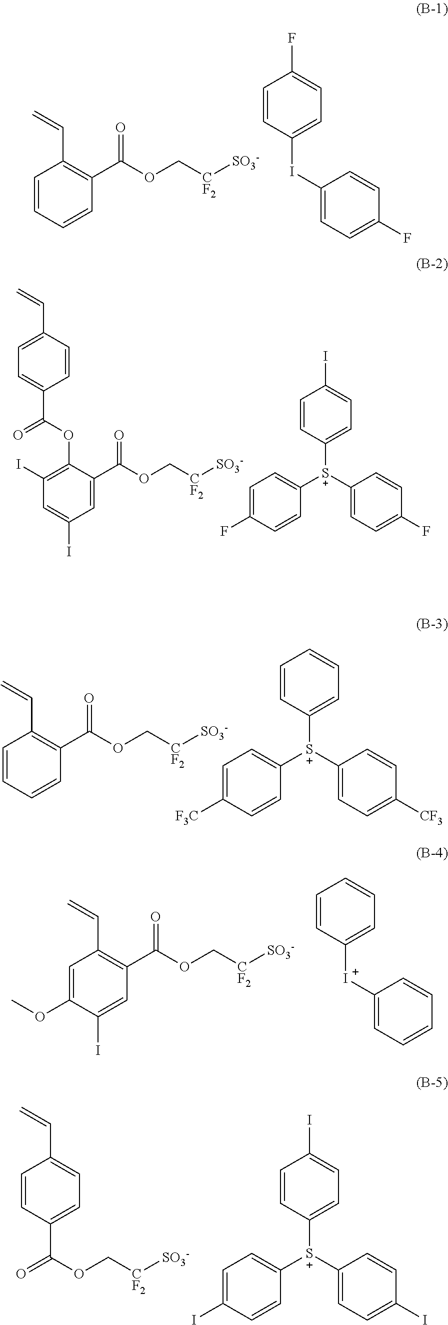

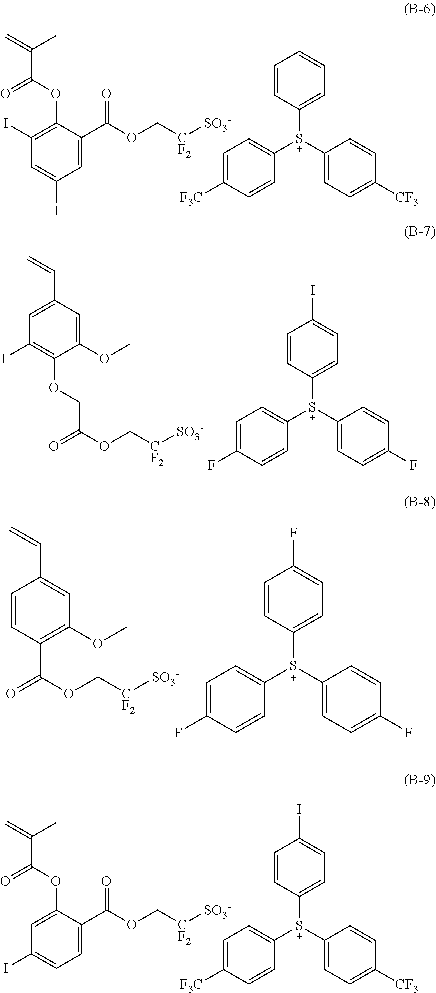

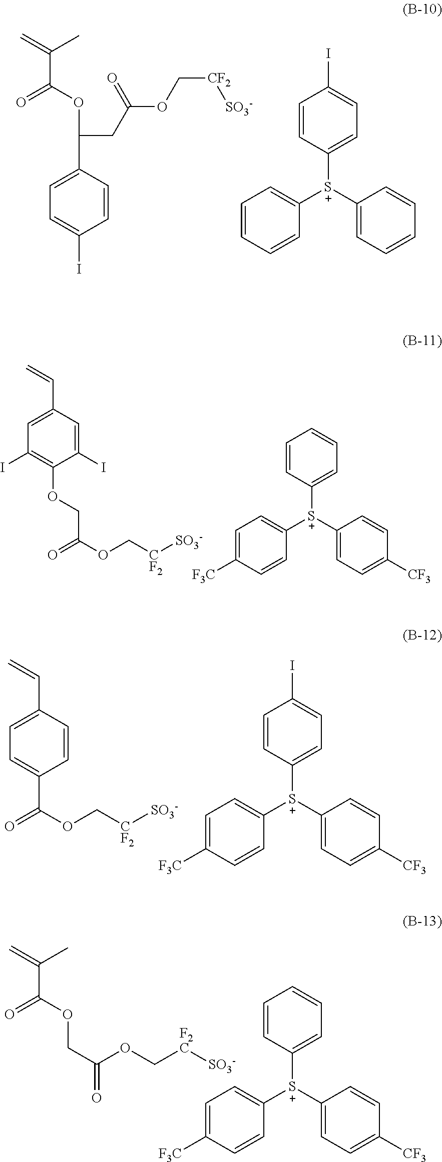

### <Synthesis of Polymer>

A method for measuring weight-average molecular weight (Mw) and number-average molecular weight (Mn) of a polymer will be described below.

### [Mw and Mn]

Mw and Mn of a polymer were measured under the following condition by gel permeation chromatography (GPC) using GPC columns manufactured by Tosoh Corporation (two columns of “SuperAW2500”, one column of “SuperAW3000”, and one column of “SuperAW4000”).
Eluent: tetrahydrofuran (manufactured by FUJIFILM Wako Pure Chemical Corporation)
Flow rate: 1.0 mL/min
Sample concentration: 1.0 mass %
Sample injection amount: 100 μL
Column temperature: 40° C.
Detector: differential refractometer
Standard substance: monodisperse polystyrene

### [Synthesis Examples P1 to P24] Synthesis of Polymers (P-1) to (P-23) and (Pc-1)

A copolymerization reaction of monomers was performed in a tetrahydrofuran (THF) solvent. The product was crystallized in methanol, and further repeatedly washed with hexane. The product was isolated and dried to obtain polymers (P-1) to (P-23) and (Pc-1) having composition described below. The composition of the obtained polymer was determined by ¹H-NMR to determine Mw and dispersion degree (Mw/Mn) under the aforementioned GPC condition. Table 1 shows the composition of monomers, Mw, and dispersion degree of the polymer obtained in each Synthesis Example. Abbreviations of the monomers are as follows.

### (Abbreviations of Monomers)

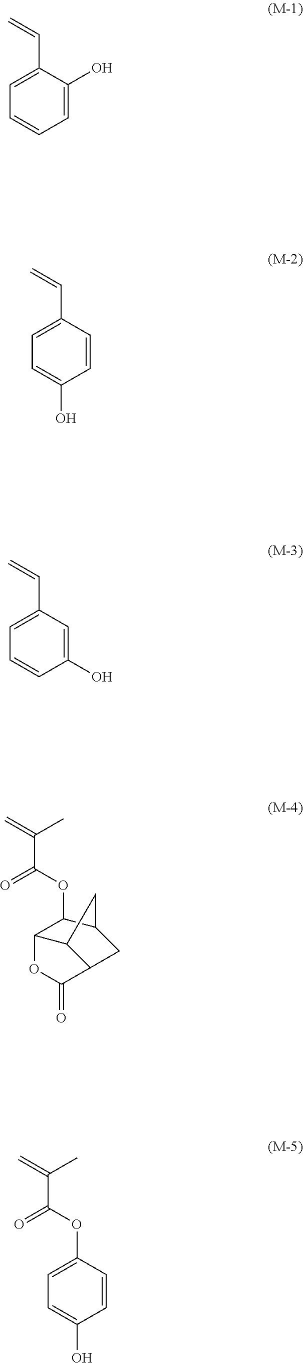

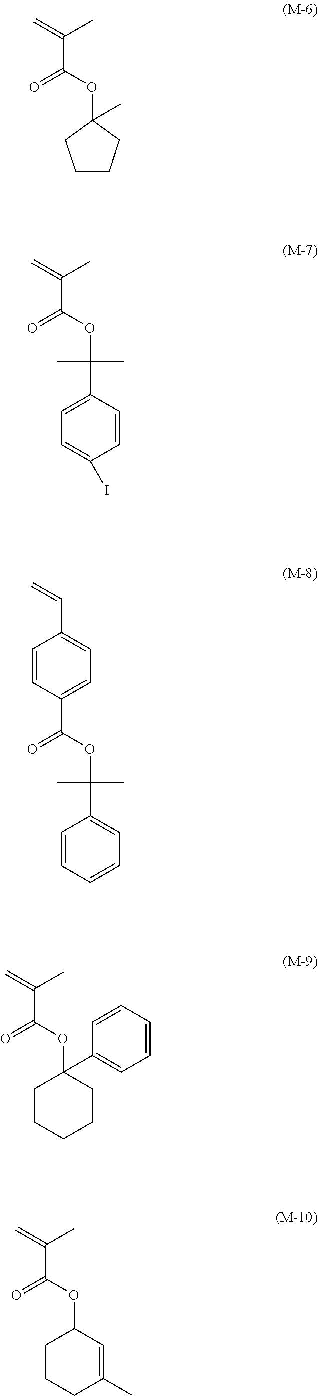

TABLE 1

| | | Monomer having onium salt | | | | | | | | | | | |
|---|---|---|---|---|---|---|---|---|---|---|---|---|---|
| | | structure | | | | | | | | | | | |
| | | Monomer to | | Monomer to | | Monomer to | | Monomer to | | Monomer to | | | |
| | | yield structural | | yield other | | yield structural | | yield structural | | yield structural | | | |
| | Polymer | unit (I) | | structural unit | | unit (II) | | unit (III) | | unit (IV) | | | |
| | (P) | Type | mol % | Type | mol % | Type | mol % | Type | mol % | Type | mol % | Mw | Mw/Mn |
| Synthesis | P-1 | B-2 | 10 | — | — | M-2 | 30 | M-6 | 60 | — | — | 8,700 | 1.7 |
| Example P1 | | | | | | | | | | | | | |
| Synthesis | P-2 | B-2 | 10 | — | — | M-2 | 30 | M-7 | 60 | — | — | 8,500 | 1.7 |
| Example P2 | | | | | | | | | | | | | |
| Synthesis | P-3 | B-2 | 10 | — | — | M-2 | 30 | M-8 | 60 | — | — | 8,400 | 1.7 |
| Example P3 | | | | | | | | | | | | | |
| Synthesis | P-4 | B-2 | 10 | — | — | M-2 | 30 | M-9 | 60 | — | — | 8,600 | 1.7 |
| Example P4 | | | | | | | | | | | | | |
| Synthesis | P-5 | B-2 | 10 | — | — | M-2 | 30 | M-10 | 60 | — | — | 8,700 | 1.7 |
| Example P5 | | | | | | | | | | | | | |
| Synthesis | P-6 | B-2 | 10 | — | — | M-2/M-1 | 20/10 | M-6 | 60 | — | — | 8,300 | 1.7 |
| Example P6 | | | | | | | | | | | | | |
| Synthesis | P-7 | B-2 | 10 | — | — | M-2/M-3 | 20/10 | M-6 | 60 | — | — | 8,700 | 1.7 |
| Example P7 | | | | | | | | | | | | | |
| Synthesis | P-8 | B-2 | 10 | — | — | M-2 | 20 | M-6 | 60 | M-4 | 10 | 8,400 | 1.7 |
| Example P8 | | | | | | | | | | | | | |
| Synthesis | P-9 | B-2 | 10 | — | — | M-2/M-5 | 20/10 | M-6 | 60 | — | — | 8,500 | 1.7 |
| Example P9 | | | | | | | | | | | | | |
| Synthesis | P-10 | B-2 | 10 | — | — | M-2 | 35 | M-6 | 55 | — | — | 8,100 | 1.7 |
| Example P10 | | | | | | | | | | | | | |
| Synthesis | P-11 | B-2 | 10 | — | — | M-2 | 40 | M-6 | 50 | — | — | 8,200 | 1.7 |
| Example P11 | | | | | | | | | | | | | |
| Synthesis | P-12 | B-1 | 10 | — | — | M-2 | 30 | M-6 | 60 | — | — | 8,300 | 1.7 |
| Example P12 | | | | | | | | | | | | | |
| Synthesis | P-13 | B-3 | 10 | — | — | M-2 | 30 | M-6 | 60 | — | — | 8,100 | 1.7 |
| Example P13 | | | | | | | | | | | | | |
| Synthesis | P-14 | B-4 | 10 | — | — | M-2 | 30 | M-6 | 60 | — | — | 8,200 | 1.7 |
| Example P14 | | | | | | | | | | | | | |
| Synthesis | P-15 | B-5 | 10 | — | — | M-2 | 30 | M-6 | 60 | — | — | 8,800 | 1.7 |
| Example P15 | | | | | | | | | | | | | |
| Synthesis | P-16 | B-6 | 10 | — | — | M-2 | 30 | M-6 | 60 | — | — | 8,900 | 1.7 |
| Example P16 | | | | | | | | | | | | | |
| Synthesis | P-17 | B-7 | 10 | — | — | M-2 | 30 | M-6 | 60 | — | — | 8,800 | 1.7 |
| Example P17 | | | | | | | | | | | | | |
| Synthesis | P-18 | B-8 | 10 | — | — | M-2 | 30 | M-6 | 60 | — | — | 8,700 | 1.7 |
| Example P18 | | | | | | | | | | | | | |
| Synthesis | P-19 | B-9 | 10 | — | — | M-2 | 30 | M-6 | 60 | — | — | 8,100 | 1.7 |
| Example P19 | | | | | | | | | | | | | |
| Synthesis | P-20 | B-10 | 10 | — | — | M-2 | 30 | M-6 | 60 | — | — | 8,000 | 1.7 |
| Example P20 | | | | | | | | | | | | | |
| Synthesis | P-21 | B-11 | 10 | — | — | M-2 | 30 | M-6 | 60 | — | — | 8,200 | 1.7 |
| Example P21 | | | | | | | | | | | | | |
| Synthesis | P-22 | B-12 | 10 | — | — | M-2 | 30 | M-6 | 60 | — | — | 8,600 | 1.7 |
| Example P22 | | | | | | | | | | | | | |
| Synthesis | P-23 | B-8 | 10 | — | — | M-2 | 30 | M-7 | 60 | — | — | 8,400 | 1.7 |
| Example P23 | | | | | | | | | | | | | |
| Synthesis | Pc-1 | — | — | B-13 | 10 | M-2 | 30 | M-6 | 60 | — | — | 8,600 | 1.7 |
| Example P24 | | | | | | | | | | | | | |

### <Preparation of Radiation-Sensitive Composition>

Radiation-sensitive compositions were prepared by using the polymers obtained in Synthesis Examples P1 to P24, the radiation-sensitive acid generators obtained in Synthesis Examples A1 to A12, acid-diffusion inhibitors, and solvents. The acid-diffusion inhibitors and the solvents used for preparing the radiation-sensitive composition will be described below.

### (Acid-Diffusion Inhibitor)

Compounds (D-1) to (D-8): Compounds represented by the following formulae (D-1) to (D-8)

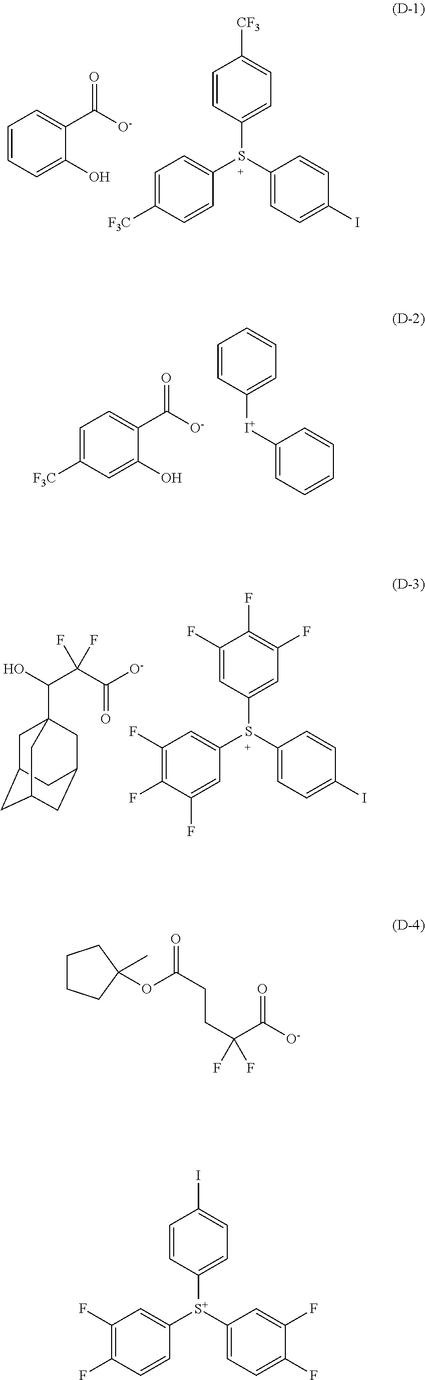

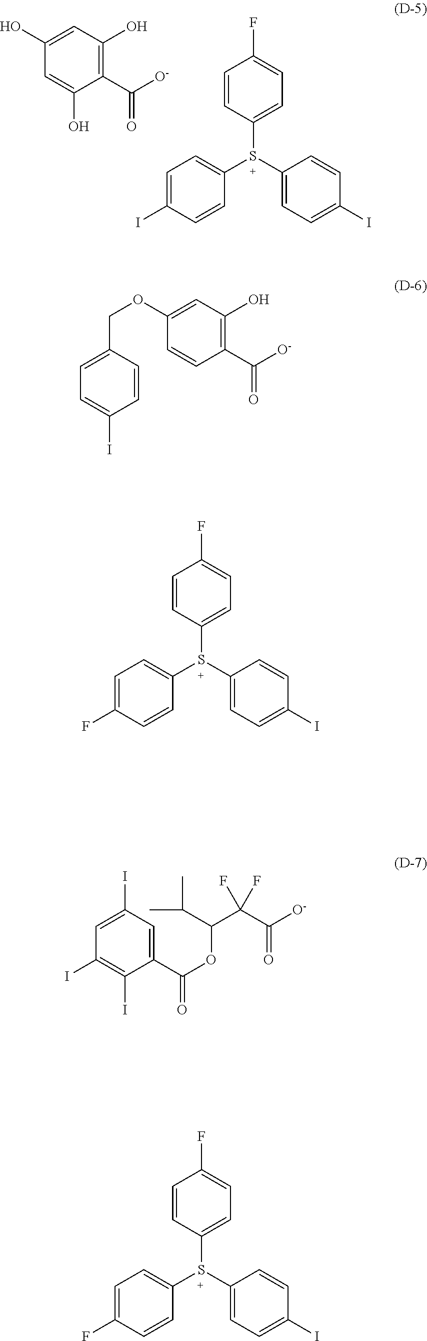

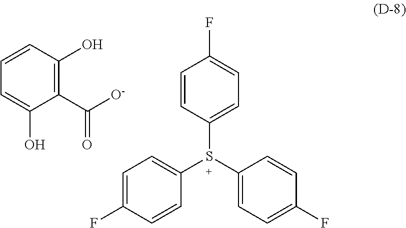

### (Solvent)

PGMEA: Propylene glycol monomethyl ether acetate
GBL: γ-Butyrolactone
CHN: Cyclohexanone
PGME: Propylene glycol monomethyl ether
DAA: Diacetone alcohol
EL: Ethyl lactate
HBM: Methyl 2-hydroxyisobutyrate

### Example 1

Blending 100 parts by mass of the polymer (P-1), 7.5 parts by mass of the compound (A-1) as the radiation-sensitive acid generator, the compound (D-8) as the acid-diffusion inhibitor at 43 mol % relative to a total of anions in the polymer (P-1) and the compound (A-1), and 5,500 parts by mass of PGMEA and 1,500 parts by mass of DAA as the solvent was performed. This mixture was filtered with a membrane filter having a pore diameter of 0.2 μm to prepare a radiation-sensitive composition (R-1).

### Examples 2 to 53 and Comparative Examples 1 to 6

Radiation-sensitive compositions (R-2) to (R-53) and (CR-1) to (CR-6) were prepared in the same manner as in Example 1 except that components with types at blending amounts described in Tables 2 and 3 were used.

TABLE 2

| | | Polymer | | Acid generator | | Acid-diffusion | | Solvent | |
|---|---|---|---|---|---|---|---|---|---|
| | Radiation- | | Content | | Content | inhibitor | | | Content |
| | sensitive | | (part(s) | | (part(s) | | Content | | (part(s) |
| | composition | Type | by mass) | Type | by mass) | Type | (mol %) | Type | by mass) |
| Example 1 | R-1 | P-1 | 100 | A-1 | 7.5 | D-8 | 43 | PGMEA/DAA | 5500/1500 |
| Example 2 | R-2 | P-2 | 100 | A-1 | 7.5 | D-8 | 43 | PGMEA/DAA | 5500/1500 |
| Example 3 | R-3 | P-3 | 100 | A-1 | 7.5 | D-8 | 43 | PGMEA/DAA | 5500/1500 |
| Example 4 | R-4 | P-4 | 100 | A-1 | 7.5 | D-8 | 43 | PGMEA/DAA | 5500/1500 |
| Example 5 | R-5 | P-5 | 100 | A-1 | 7.5 | D-8 | 43 | PGMEA/DAA | 5500/1500 |
| Example 6 | R-6 | P-6 | 100 | A-1 | 7.5 | D-8 | 43 | PGMEA/DAA | 5500/1500 |
| Example 7 | R-7 | P-7 | 100 | A-1 | 7.5 | D-8 | 43 | PGMEA/DAA | 5500/1500 |
| Example 8 | R-8 | P-8 | 100 | A-1 | 7.5 | D-8 | 43 | PGMEA/DAA | 5500/1500 |
| Example 9 | R-9 | P-9 | 100 | A-1 | 7.5 | D-8 | 43 | PGMEA/DAA | 5500/1500 |
| Example 10 | R-10 | P-10 | 100 | A-1 | 7.5 | D-8 | 43 | PGMEA/DAA | 5500/1500 |
| Example 11 | R-11 | P-11 | 100 | A-1 | 7.5 | D-8 | 43 | PGMEA/DAA | 5500/1500 |
| Example 12 | R-12 | P-12 | 100 | A-1 | 7.5 | D-8 | 43 | PGMEA/DAA | 5500/1500 |
| Example 13 | R-13 | P-13 | 100 | A-1 | 7.5 | D-8 | 43 | PGMEA/DAA | 5500/1500 |
| Example 14 | R-14 | P-14 | 100 | A-1 | 7.5 | D-8 | 43 | PGMEA/DAA | 5500/1500 |
| Example 15 | R-15 | P-15 | 100 | A-1 | 7.5 | D-8 | 43 | PGMEA/DAA | 5500/1500 |
| Example 16 | R-16 | P-16 | 100 | A-1 | 7.5 | D-8 | 43 | PGMEA/DAA | 5500/1500 |
| Example 17 | R-17 | P-17 | 100 | A-1 | 7.5 | D-8 | 43 | PGMEA/DAA | 5500/1500 |
| Example 18 | R-18 | P-18 | 100 | A-1 | 7.5 | D-8 | 43 | PGMEA/DAA | 5500/1500 |
| Example 19 | R-19 | P-19 | 100 | A-1 | 7.5 | D-8 | 43 | PGMEA/DAA | 5500/1500 |
| Example 20 | R-20 | P-20 | 100 | A-1 | 7.5 | D-8 | 43 | PGMEA/DAA | 5500/1500 |
| Example 21 | R-21 | P-21 | 100 | A-1 | 7.5 | D-8 | 43 | PGMEA/DAA | 5500/1500 |
| Example 22 | R-22 | P-1 | 100 | — | — | D-8 | 43 | PGMEA/DAA | 5500/1500 |
| Example 23 | R-23 | P-14 | 100 | — | — | D-8 | 43 | PGMEA/DAA | 5500/1500 |
| Example 24 | R-24 | P-15 | 100 | — | — | D-8 | 43 | PGMEA/DAA | 5500/1500 |
| Example 25 | R-25 | P-16 | 100 | — | — | D-8 | 43 | PGMEA/DAA | 5500/1500 |
| Example 26 | R-26 | P-17 | 100 | — | — | D-8 | 43 | PGMEA/DAA | 5500/1500 |
| Example 27 | R-27 | P-19 | 100 | — | — | D-8 | 43 | PGMEA/DAA | 5500/1500 |
| Example 28 | R-28 | P-20 | 100 | — | — | D-8 | 43 | PGMEA/DAA | 5500/1500 |
| Example 29 | R-29 | P-21 | 100 | — | — | D-8 | 43 | PGMEA/DAA | 5500/1500 |
| Example 30 | R-30 | P-1 | 100 | A-2 | 7.5 | D-8 | 43 | PGMEA/DAA | 5500/1500 |

TABLE 3

| | | Polymer | | Acid generator | | Acid-diffusion | | Solvent | |
|---|---|---|---|---|---|---|---|---|---|
| | Radiation- | | Content | | Content | inhibitor | | | Content |
| | sensitive | | (part(s) | | (part(s) | | Content | | (part(s) |
| | composition | Type | by mass) | Type | by mass) | Type | (mol %) | Type | by mass) |
| Example 31 | R-31 | P-1 | 100 | A-3 | 7.5 | D-8 | 43 | PGMEA/DAA | 5500/1500 |
| Example 32 | R-32 | P-1 | 100 | A-4 | 7.5 | D-8 | 43 | PGMEA/DAA | 5500/1500 |
| Example 33 | R-33 | P-1 | 100 | A-5 | 7.5 | D-8 | 43 | PGMEA/DAA | 5500/1500 |
| Example 34 | R-34 | P-1 | 100 | A-6 | 7.5 | D-8 | 43 | PGMEA/DAA | 5500/1500 |
| Example 35 | R-35 | P-1 | 100 | A-7 | 7.5 | D-8 | 43 | PGMEA/DAA | 5500/1500 |
| Example 36 | R-36 | P-1 | 100 | A-8 | 7.5 | D-8 | 43 | PGMEA/DAA | 5500/1500 |
| Example 37 | R-37 | P-1 | 100 | A-9 | 7.5 | D-8 | 43 | PGMEA/DAA | 5500/1500 |
| Example 38 | R-38 | P-1 | 100 | A-10 | 7.5 | D-8 | 43 | PGMEA/DAA | 5500/1500 |
| Example 39 | R-39 | P-1 | 100 | A-11 | 7.5 | D-8 | 43 | PGMEA/DAA | 5500/1500 |
| Example 40 | R-40 | P-1 | 100 | A-1 | 7.5 | D-1 | 43 | PGMEA/DAA | 5500/1500 |
| Example 41 | R-41 | P-1 | 100 | A-1 | 7.5 | D-2 | 43 | PGMEA/DAA | 5500/1500 |
| Example 42 | R-42 | P-1 | 100 | A-1 | 7.5 | D-3 | 43 | PGMEA/DAA | 5500/1500 |
| Example 43 | R-43 | P-1 | 100 | A-1 | 7.5 | D-4 | 43 | PGMEA/DAA | 5500/1500 |
| Example 44 | R-44 | P-1 | 100 | A-1 | 7.5 | D-5 | 43 | PGMEA/DAA | 5500/1500 |
| Example 45 | R-45 | P-1 | 100 | A-1 | 7.5 | D-6 | 43 | PGMEA/DAA | 5500/1500 |
| Example 46 | R-46 | P-1 | 100 | A-1 | 7.5 | D-7 | 43 | PGMEA/DAA | 5500/1500 |
| Example 47 | R-47 | P-1 | 100 | A-1 | 7.5 | D-8 | 43 | PGMEA/GBL | 5500/900 |
| Example 48 | R-48 | P-1 | 100 | A-1 | 7.5 | D-8 | 43 | PGMEA/PGME | 5500/1500 |
| Example 49 | R-49 | P-1 | 100 | A-1 | 7.5 | D-8 | 43 | PGMEA/CHN | 5500/1500 |
| Example 50 | R-50 | P-1 | 100 | A-1 | 7.5 | D-8 | 43 | PGMEA/EL | 5500/1500 |
| Example 51 | R-51 | P-1 | 100 | A-1 | 7.5 | D-8 | 43 | PGMEA/HBM | 5500/1500 |
| Example 52 | R-52 | P-1 | 100 | A-1 | 10 | D-1 | 43 | PGMEA/DAA | 5500/1500 |
| Example 53 | R-53 | P-1 | 100 | A-1 | 15 | D-1 | 43 | PGMEA/DAA | 5500/1500 |
| Comparative | CR-1 | Pc-1 | 100 | A-1 | 7.5 | D-8 | 43 | PGMEA/DAA | 5500/1500 |
| Example 1 | | | | | | | | | |
| Comparative | CR-2 | Pc-1 | 100 | — | — | D-8 | 43 | PGMEA/DAA | 5500/1500 |
| Example 2 | | | | | | | | | |
| Comparative | CR-3 | P-13 | 100 | — | — | D-8 | 43 | PGMEA/DAA | 5500/1500 |
| Example 3 | | | | | | | | | |
| Comparative | CR-4 | P-22 | 100 | — | — | D-8 | 43 | PGMEA/DAA | 5500/1500 |
| Example 4 | | | | | | | | | |
| Comparative | CR-5 | P-23 | 100 | — | — | D-8 | 43 | PGMEA/DAA | 5500/1500 |
| Example 5 | | | | | | | | | |
| Comparative | CR-6 | P-18 | 100 | A-12 | 7.5 | D-8 | 43 | PGMEA/DAA | 5500/1500 |
| Example 6 | | | | | | | | | |

### <Formation of Resist Pattern>

On a 12-inch silicon wafer surface on which an underlayer film (AL412 (manufactured by Brewer Science, Inc.)) with 20 nm in film thickness was formed, each of the radiation-sensitive compositions was applied by using a spin coater (“CLEAN TRACK ACT12”, Tokyo Electron Ltd.). Soft baking (SB) was performed at 100° C. for 60 seconds, and then the composition was cooled at 23° C. for 30 seconds to form a resist film with 35 nm in film thickness. Then, this resist film was irradiated with EUV light by using an EUV exposure apparatus (model: “NXE3300”, manufactured by ASML Holding N.V., NA=0.33, illumination condition: Conventional s=0.89). The resist film after the irradiation with EUV light was subjected to post exposure baking (PEB) at 100° C. for 60 seconds. Subsequently, the resist film was developed by using a TMAH aqueous solution at 2.38 mass % at 23° C. for 30 seconds to form a positive-type 25-nm contact-hole pattern with 50-nm pitch.

### <Evaluation>

Each of the resist patterns formed by the above method was measured according to the following methods to evaluate sensitivity, CDU performance, and development-defect inhibition of each of the radiation-sensitive compositions. For measuring a length of the resist pattern, a scanning electron microscope (“CG-5000”, Hitachi High-Tech Corporation) was used. Tables 4 and 5 show the evaluation results.

### [Sensitivity]

In forming the resist pattern, an exposure dose for forming the 25-nm contact-hole pattern was specified as an optimal exposure dose, and this optimal exposure dose was specified as sensitivity (mJ/cm²). A smaller value thereof indicates better sensitivity. A case where the sensitivity was less than 48 mJ/cm² was judged as “A” (extremely good), a case where the sensitivity was 48 mJ/cm² or more and 51 mJ/cm² or less was judged as “B” (good), and a case where the sensitivity was more than 51 mJ/cm² was judged as “C” (poor).

### [CUD Performance]

The 25-nm contact-hole pattern was observed from above by using the above scanning electron microscope, and the length was measured at given 800 points in total. Variation of size (3a) was determined, and this variation was specified as CDU (nm). A lower CDU value indicates smaller variation of the hole diameter in a long period (namely, variation of diameter among different holes) and better CDU performance. A case where the CDU was less than 3.4 nm was judged as “A” (extremely good), a case where the CDU was 3.4 nm or more and less than 3.6 nm was judged as “B” (good), and a case where the CDU was more than 3.6 nm was judged as “C” (poor).

### [Development-Defect Inhibition]

The resist film was exposed at the optimal exposure dose, and developed to form a 25-nm contact hole pattern. A number of defects on this wafer for defect inspection was measured by using a defect inspection apparatus (“KLA2810”, KLA-Tencor Corporation). The observed defects were classified into those judged to be derived from the resist film and foreign matters derived from an external environment. In evaluation of the development defects, a case where the number of the defects judged to be derived from the resist film was less than 35 was judged as “A” (extremely good), a case where the number was 35 or more and 60 or less was judged as “B” (good), and a case where the number was more than 60 was judged as “C” (poor).

TABLE 4

| | Radiation- | | | Development- |
|---|---|---|---|---|
| | sensitive | | CUD | Defect |
| | composition | Sensitivity | Performance | Inhibition |
| Example 1 | R-1 | A | A | A |
| Example 2 | R-2 | A | A | A |
| Example 3 | R-3 | A | A | A |
| Example 4 | R-4 | A | A | A |
| Example 5 | R-5 | A | A | A |
| Example 6 | R-6 | A | A | A |
| Example 7 | R-7 | A | A | A |
| Example 8 | R-8 | A | A | A |
| Example 9 | R-9 | A | A | A |
| Example 10 | R-10 | B | A | A |
| Example 11 | R-11 | B | A | A |
| Example 12 | R-12 | A | B | A |
| Example 13 | R-13 | B | B | B |
| Example 14 | R-14 | A | A | A |
| Example 15 | R-15 | A | A | A |
| Example 16 | R-16 | A | A | A |
| Example 17 | R-17 | A | A | A |
| Example 18 | R-18 | B | B | B |
| Example 19 | R-19 | A | A | B |
| Example 20 | R-20 | B | A | A |
| Example 21 | R-21 | A | A | A |
| Example 22 | R-22 | A | A | A |
| Example 23 | R-23 | A | A | A |
| Example 24 | R-24 | A | A | A |
| Example 25 | R-25 | A | A | A |
| Example 26 | R-26 | A | A | A |
| Example 27 | R-27 | A | A | B |
| Example 28 | R-28 | A | A | A |
| Example 29 | R-29 | A | A | A |
| Example 30 | R-30 | A | A | A |

TABLE 5

| | Radiation- | | | Development- |
|---|---|---|---|---|
| | sensitive | | CUD | Defect |
| | composition | Sensitivity | Performance | Inhibition |
| Example 31 | R-31 | A | A | A |
| Example 32 | R-32 | A | A | A |
| Example 33 | R-33 | A | A | A |
| Example 34 | R-34 | A | B | A |
| Example 35 | R-35 | B | A | A |
| Example 36 | R-36 | A | A | A |
| Example 37 | R-37 | A | A | A |
| Example 38 | R-38 | A | B | B |
| Example 39 | R-39 | B | A | B |
| Example 40 | R-40 | A | A | A |
| Example 41 | R-41 | A | A | A |
| Example 42 | R-42 | A | A | A |
| Example 43 | R-43 | A | A | A |
| Example 44 | R-44 | A | A | A |
| Example 45 | R-45 | A | A | A |
| Example 46 | R-46 | A | A | A |
| Example 47 | R-47 | A | A | A |
| Example 48 | R-48 | A | A | A |
| Example 49 | R-49 | A | A | A |
| Example 50 | R-50 | A | A | A |
| Example 51 | R-51 | A | A | A |
| Example 52 | R-52 | A | A | A |
| Example 53 | R-53 | A | A | A |
| Comparative | CR-1 | C | B | C |
| Example 1 | | | | |
| Comparative | CR-2 | C | C | C |
| Example 2 | | | | |
| Comparative | CR-3 | C | C | B |
| Example 3 | | | | |
| Comparative | CR-4 | C | C | B |
| Example 4 | | | | |
| Comparative | CR-5 | C | B | B |
| Example 5 | | | | |
| Comparative | CR-6 | C | C | C |
| Example 6 | | | | |

As shown in Tables 4 and 5, all the radiation-sensitive compositions of Examples 1 to 53 improved sensitivity, CDU performance, and development-defect inhibition with good balance. In contrast, the radiation-sensitive compositions of Comparative Examples 1 and 2, which used the polymer without specific partial structure instead of the polymer (P) having the specific partial structure exhibited evaluation of “C” (poor) in sensitivity and development-defect inhibition. The radiation-sensitive composition of Comparative Example 2, which contained no acid generator, exhibited “C” (poor) also in CDU performance. The radiation-sensitive compositions of Comparative Examples 3 to 6, which contained the polymer (P) having the specific partial structure but satisfied none of the requirements 1 to 3, exhibited evaluation of “C” (poor) in one or more of sensitivity, CDU performance, and development-defect inhibition.

From the above results, it can be said that the radiation-sensitive composition and the method for forming a resist pattern of the present disclosure can improve sensitivity, CDU performance, and development-defect inhibition with good balance. Therefore, the radiation-sensitive composition and the method for forming a resist pattern of the present disclosure are suitable for forming a fine resist pattern in lithography process for electronic devices such as semiconductor devices and liquid crystal devices.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive composition, comprising:
a polymer comprising a partial structure represented by formula (1): 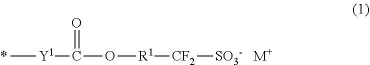
wherein Y¹ represents a divalent group represented by formula (2-1) or formula (2-2): 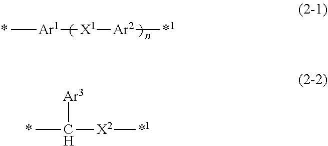
wherein Ar¹ represents a divalent aromatic ring group, Ar² represents a single bond or a divalent aromatic ring group, Ar³ represents a monovalent aromatic ring group, X¹ and X² each independently represent a divalent linking group, n represents 0 or 1, *¹ represents an attachment point to the carbonyl group in the formula (1), and * represents an attachment point,
R¹ represents a divalent hydrocarbon group, M⁺ represents a sulfonium cation or an iodonium cation, and * represents an attachment point; and
a radiation-sensitive acid generating substance, wherein
the radiation-sensitive composition satisfies one or more of the following requirement 1, requirement 2, and requirement 3:
requirement 1: the partial structure represented by the formula (1) comprises two or more iodine atoms;
requirement 2: the radiation-sensitive acid generating substance comprises an onium salt comprising two or more iodine atoms;
requirement 3: the partial structure represented by the formula (1) comprises an iodine atom, and the radiation-sensitive acid generating substance comprises an onium salt comprising an iodine atom.

2. The radiation-sensitive composition according to claim 1, wherein the polymer comprises a structural unit (I) comprising the partial structure represented by the formula (1).

3. The radiation-sensitive composition according to claim 2, wherein the structural unit (I) is represented by formula (3): 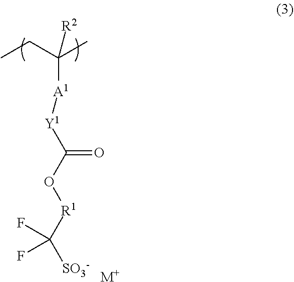
wherein R² represents a hydrogen atom, a fluorine atom, a methyl group, or a trifluoromethyl group; A¹ represents a single bond or *²—CO—O—, wherein *² represents an attachment point to a main chain of the polymer; and Y¹, R¹, and M⁺ are as defined in the formula (1).

4. The radiation-sensitive composition according to claim 1, wherein when the partial structure represented by the formula (1) or the radiation-sensitive acid generating substance comprises an iodine atom, the iodine is bonded to an aromatic ring.

5. The radiation-sensitive composition according to claim 1, wherein the polymer further comprises a structural unit comprising an aromatic ring and a hydroxy group bonded to the aromatic ring.

6. The radiation-sensitive composition according to claim 1, wherein the polymer further comprises a structural unit comprising an acid-dissociable group.

7. The radiation-sensitive composition according to claim 1, wherein the radiation-sensitive acid generating substance comprises a photodegradable base.

8. A method for forming a resist pattern, comprising:
forming a resist film on a substrate by applying the radiation-sensitive composition according to claim 1;
exposing the resist film; and
developing the exposed resist film.

9. A compound represented by formula (3-1): 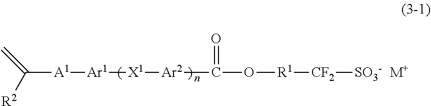
wherein R² represents a hydrogen atom, a fluorine atom, a methyl group, or a trifluoromethyl group; A¹ represents a single bond or *³—CO—O—; *³ represents an attachment point to the carbon atom to which R² is bonded; Ar¹ represents a divalent aromatic ring group; Ar² represents a single bond or a divalent aromatic ring group; X¹ represents a divalent linking group; n represents 0 or 1; R¹ represents a divalent hydrocarbon group; and M⁺ represents a sulfonium cation or an iodonium cation,
wherein the compound comprises two or more iodine atoms in the formula (3-1).

10. A compound represented by the following formula (3-2): 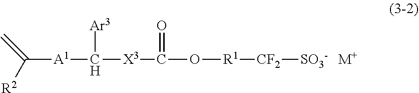
wherein R² represents a hydrogen atom, a fluorine atom, a methyl group, or a trifluoromethyl group; A¹ represents a single bond or *³—CO—O—; *³ represents an attachment point to the carbon atom to which R² is bonded; Ar³ represents a monovalent aromatic ring group; X² represents a divalent linking group; R¹ represents a divalent hydrocarbon group; and M⁺ represents a sulfonium cation or an iodonium cation.
